(19) United States
(12) Patent Application Publication
(10) Pub. No.: US 2005/0209336 A1
(43) Pub. Date: Sep. 22, 2005

(54) EP2 receptor agonists

(76) Inventors: Richard Anthony Borman, Royston (GB); Robert Alexander Coleman, Royston (GB); Kenneth Lyle Clark, Royston (GB); Keith Mills, Royston (GB); Alexander William Oxford, Royston (GB); Jian Gang Zhang, Royston (GB); Peter Thomas Duff, Southampton (GB)

Correspondence Address: NIXON & VANDERHYE, PC, 901 NORTH GLEBE ROAD, 11TH FLOOR, ARLINGTON, VA, 22203, US

(21) Appl. No.: 11/019,133

(22) Filed: Dec. 22, 2004

Related U.S. Application Data
(60) Provisional application No. 60/531,979, filed on Dec. 24, 2003.
(60) Provisional application No. 60/622,012, filed on Oct. 27, 2004.

(30) Foreign Application Priority Data
Dec. 22, 2003 (GB) 0329620.9

(51) Int. Cl. A61K 31/557; C07C 59/185

(52) U.S. Cl. 514/570; 514/573; 554/116

(57) ABSTRACT

A compound selected from one of the following: 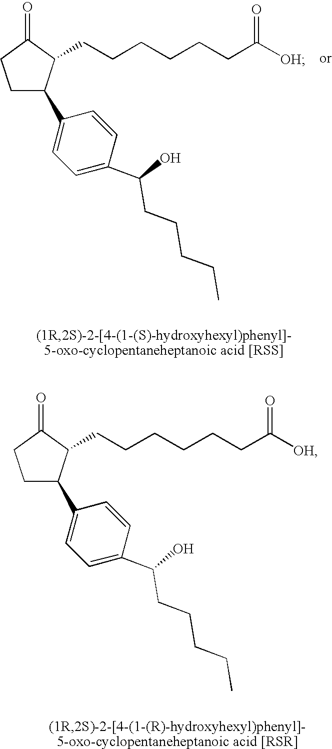
or a salt, solvate, chemically protected form or prodrug thereof, and its use in treating conditions alleviated by agonism of an EP₂ receptor.

This invention relates to certain stereoisomers of AH13205, (±)-trans-2-[4-(1-hydroxyhexyl)phenyl]-5-oxo-cyclopentaneheptanoic acid and their use as EP₂ receptor agonists. The invention also relates to pharmaceutical compositions comprising these stereoisomers, and the use of these stereoisomers and compositions to treat various diseases.

### BACKGROUND TO THE INVENTION

Prostanoids comprise prostaglandins (PGs) and thromboxanes (Txs) and their receptors fall into five different classes (DP, EP, FP, IP and TP) based on their sensitivity to the five naturally occurring prostanoids, PGD₂, PGE₂, PGF_{2α}, PGI₂ and TxA₂, respectively (Coleman, R. A., Prostanoid Receptors. IUPHAR compendium of receptor characterisation and classification, 2^{nd} edition, 338-353, ISBN 0-9533510-3-3, 2000). EP receptors (for which the endogenous ligand is PGE₂) have been subdivided into four types termed EP₁, EP₂, EP₃ and EP₄. These four types of EP receptors have been cloned and are distinct at both a molecular and pharmacological level (Coleman, R. A., 2000)

EP₂ agonists have been shown to be effective in the treatment of a number of conditions, including (but not limited to) dysmenorrhoea (WO 03/037433), pre-term labour (GB 2 293 101), glaucoma (WO 03/040126), ocular hypertension (WO 03/040126), immune disorders (WO 03/037433), osteoporosis (WO 98/27976, WO 01/46140), asthma (WO 03/037433), allergy (WO 03/037433), bone disease (WO 02/24647), fracture repair (WO 98/27976, WO 02/24647), fertility (Breyer, R. M., et al., Ann. N.Y. Acad. Sci., 905, 221-231 (2000)), male sexual dysfunction (WO 00/40248), female sexual dysfunction (U.S. Pat. No. 6,562,868), periodontal disease (WO 00/31084), gastric ulcer (U.S. Pat. No. 5,576,347) and renal disease (WO 98/34916).

One known EP₂ agonist with good selectivity is butaprost:

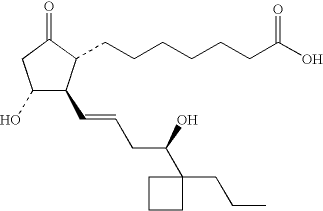

AH13205, (±)-trans-2-[4-(1-hydroxyhexyl)phenyl]-5-oxo-cyclopentaneheptanoic acid, is known as an EP₂ agonist (for example, see Hillock, C. J. and Crankshaw, D. J., European Journal of Pharmacology, 378, 99-108 (1999)).

It can also be called 7-{2-[4-(1-hydroxy-hexyl)-phenyl]-5-oxo-cyclopentyl}-heptanoic acid (fonts added for identification), and has the following structure:

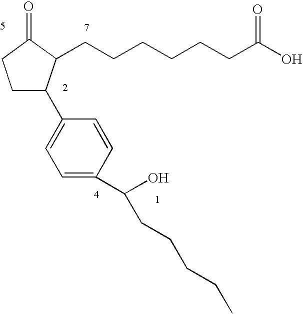

This structure has three chiral carbon atoms and hence eight possible stereoisomers. When the groups on the cyclic pentanone are in a trans relationship, this gives rise to four stereoisomers which are the major ones and when the groups are in a cis relationship, gives rise to four minor stereoisomers.

The four major stereoisomers have the following structures:

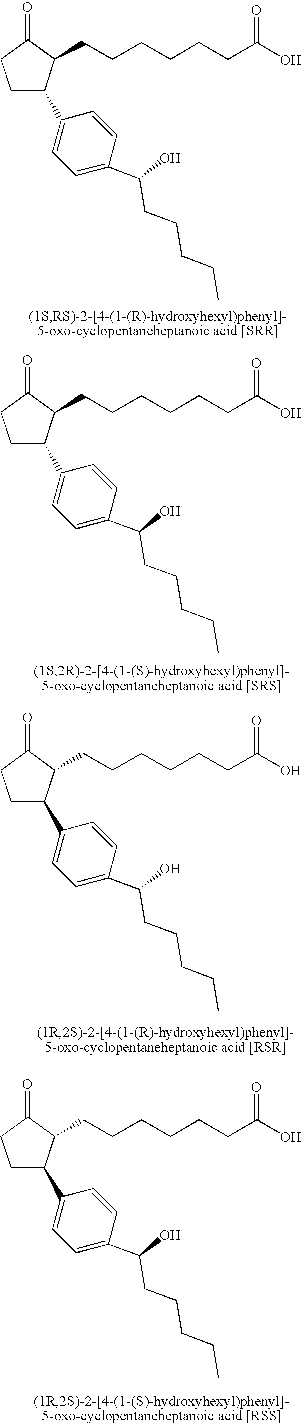

The present applicants have been able to separate the four major stereoisomers from each other and have determined their relative activities. However, initial attempts to separate these stereoisomers were not successful.

Attempts were carried out on a mixture of all the stereoisomers in their acid form using chiral HPLC using a variety of commercially available stationary phases, but these were unsuccessful.

Many attempts at separation were carried out on the two mixtures of esters produced in example 1 below, using chiral HPLC on a variety of commercially available stationary phases and mobile phases, but at best this method was successful on an analytical level and separation was not possible on a preparative scale.

Finally, however, attempts to separate the stereoisomers as esters was successful as is described below in Example 2.

The present inventors have also devised a stereoselective synthesis route for the stereoisomers of interest.

### SUMMARY OF THE INVENTION

In a first aspect, the present invention provides a compound selected from one of the following:

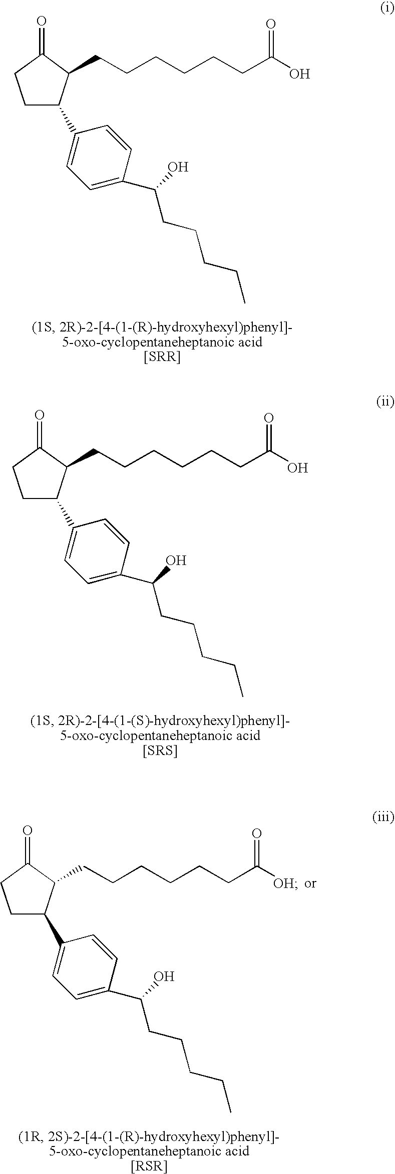

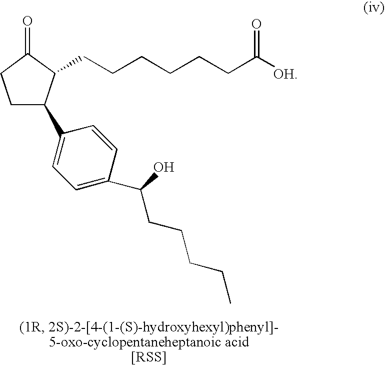

In a second aspect, the present invention provides trans-2-[4-(1-hydroxyhexyl)phenyl]-5-oxo-cyclopentaneheptanoic acid, of which at least 90% by weight is selected from one of the following forms:

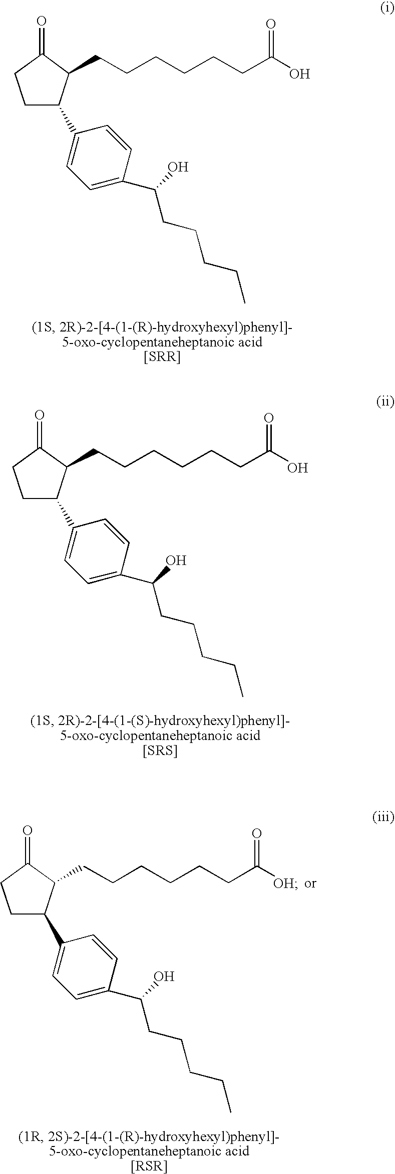

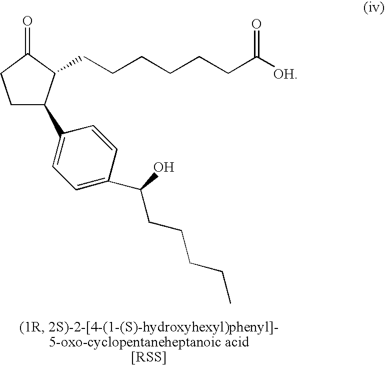

It is preferred that at least 95, 97, 99, 99.5 or 99.9% by weight of the trans-2-[4-(1-hydroxyhexyl)phenyl]-5-oxo-cyclopentaneheptanoic acid is in one of the four forms shown.

In a third aspect, the present invention provides 2-[4-(1-hydroxyhexyl)phenyl]-5-oxo-cyclopentaneheptanoic acid, of which at least 80% by weight is in one of the following forms:

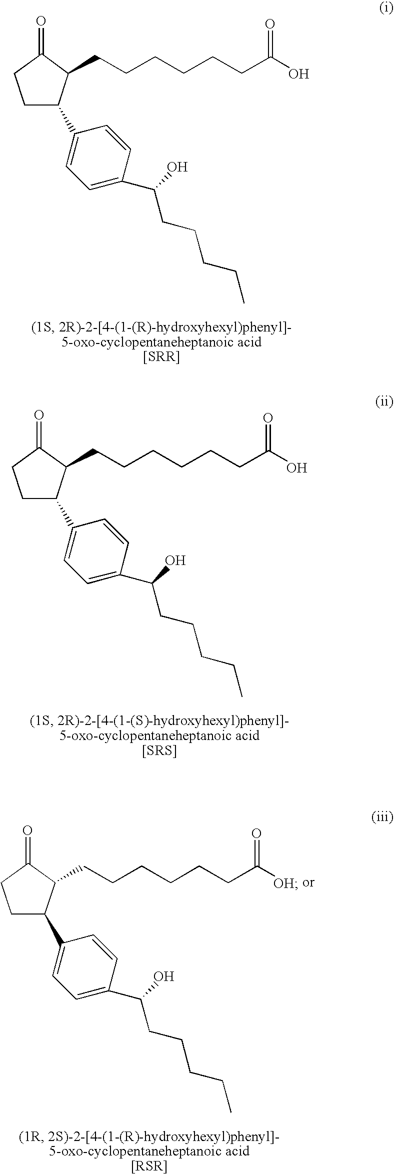

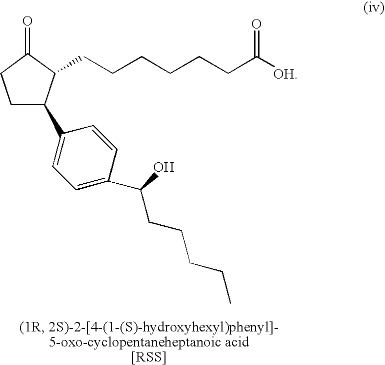

It is preferred that at least 90, 95, 97, 99, 99.5 or 99.9% by weight of the 2-[4-(1-hydroxyhexyl)phenyl]-5-oxo-cyclopentaneheptanoic acid is in one of the four forms shown.

In a fourth aspect, the invention provides a compound selected from one of the following forms:

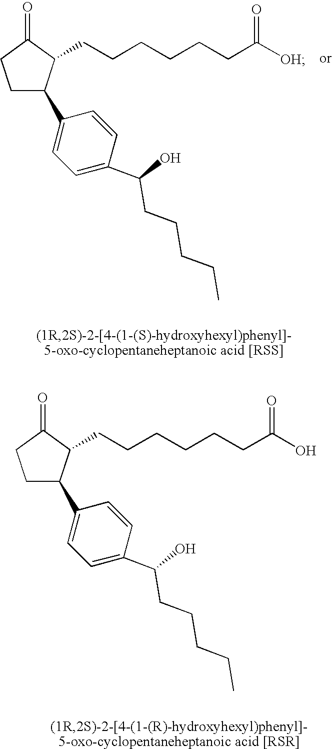

In a fifth aspect, the invention provides trans-2-[4-(1-hydroxyhexyl)phenyl]-5-oxo-cyclopentaneheptanoic acid, of which at least 90% by weight is selected from one of the following forms:

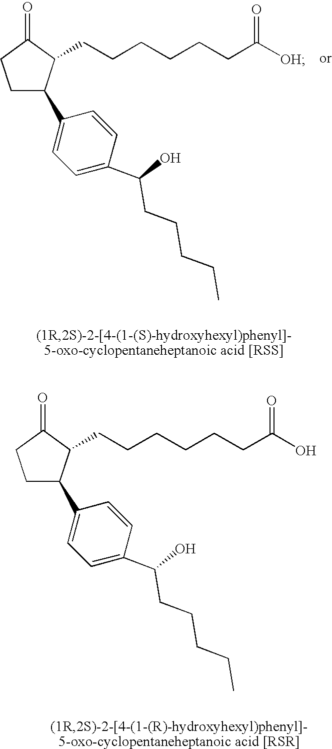

In a sixth aspect, the present invention provides 2-[4-(1-hydroxyhexyl)phenyl]-5-oxo-cyclopentaneheptanoic acid, of which at least 80% by weight is in one of the following forms:

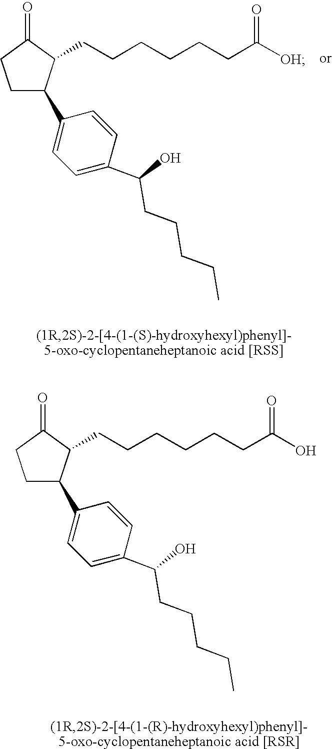

The above six aspects also relate to salts, solvates, chemically protected forms and prodrugs of the compounds described.

A seventh aspect of the invention provides a method of making a compound, comprising the following steps:
(a) asymmetrically reducing 1-(4-bromophenyl)hexan-1-one with (−)-DIP chloride to produce (S)-1-(4-bromophenyl)hexan-1-ol (S-BPH);
(b) converting the S-BPH into (S)-1-(4-bromophenyl)-1-(tert-butyldimethylsilyloxy)hexane;
(c) treating the (S)-1-(4-bromophenyl)-1-(tert-butyldimethylsilyloxy)hexane with tert-butyllithium, followed by 1:2 pentynyl copper:hexamethylphosphorus triamide, followed by condensation with 2-(6-carbomethoxyhexyl)cyclopent-2-en-1-one to produce a diastereomeric mixture of trans and cis-2-{4-[1-(S)-(tert-butyldimethylsilyloxy)hexyl]phenyl}-5-oxo-cyclopentaneheptanoic acid, methyl ester;
(d) deprotecting the t-butyldimethyl silyl group to give a diastereomeric mixture of trans and cis-2-[4-(1-(S)-hydroxyhexyl)phenyl]-5-oxo-cyclopentaneheptanoic acid, methyl ester;
(e) subjecting the diastereomeric mixture to HPLC on a chiral stationary phase, which is amylose tris(3,5-dimethylphenyl-carbamate adsorbed on a macroporous silica gel support that had been treated with 3-aminopropyl triethoxysilane in benzene, using a mobile phase of 100% ethanol;
(f) substantially isolating a single stereoisomer, being a fraction in the eluent.

An eighth aspect of the invention provides a method of making a compound, comprising the following steps:
(a) asymmetrically reducing 1-(4-bromophenyl)hexan-1-one with (+)-DIP chloride to produce (R)-1-(4-bromophenyl)hexan-1-ol (R-BPH);
(b) converting the R-BPH into (R)-1-(4-bromophenyl)-1-(tert-butyldimethylsilyloxy)hexane;
(c) treating the (R)-1-(4-bromophenyl)-1-(tert-butyldimethylsilyloxy)hexane with tert-butyllithium, followed by 1:2 pentynyl copper:hexamethylphosphorus triamide, followed by condensation with 2-(6-carbomethoxyhexyl)cyclopent-2-en-1-one to produce a diastereomeric mixture of trans and cis-2-{4-[1-(R)-(tert-butyldimethylsilyloxy)hexyl]phenyl}-5-oxo-cyclopentaneheptanoic acid, methyl ester;
(d) deprotecting the t-butyldimethyl silyl group to give a diastereomeric mixture of trans and cis-2-[4-(1-(R)-hydroxyhexyl)phenyl]-5-oxo-cyclopentaneheptanoic acid, methyl ester;
(e) subjecting the diastereomeric mixture to HPLC on a chiral stationary phase, which is amylose tris(3,5-dimethylphenyl-carbamate adsorbed on a macroporous silica gel support that had been treated with 3-aminopropyl triethoxysilane in benzene, using a mobile phase of 100% ethanol;
(f) substantially isolating a single stereoisomer, being a fraction in the eluent.

In the seventh and eighth aspects, the term “substantially” means that the compound produced is at least 90% by weight of a single stereoisomer of a compound. Preferably the compound produced is 95, 97, 99, 99.5 or 99.9% by weight of a single stereoisomer of a compound.

A ninth aspect of the invention provides a method of making a compound comprising the following steps:
(a) asymmetric addition of 1-(4-bromophenyl)-1-(tert-butyldimethylsilyloxy)hexane to 5-oxo-cyclopentenecarboxylic acid, {3-[N-benzenesulfonyl-N-(3,5-dimethylphenyl)amino]-2-bornyl}ester in the presence of an organo-copper agent and an organolithium agent to give the 1,2-trans product;
(b) conversion of the 3-[N-benzenesulphonyl-N-(3,5-dimethylphenyl)-amino]-2-bornyl group to a methyl group by reaction with methanol to give 2-{4-[1-(tert-butyldimethylsilyloxy)hexyl]phenyl}-5-oxo-cyclopentanecarboxylic acid, methyl ester;
(c) treating the 2-{4-[1-(tert-butyldimethylsilyloxy)hexyl]phenyl}-5-oxo-cyclopentanecarboxylic acid, methyl ester with ethyl-7-bromoheptanoate in the presence of base to give 1-methoxycarbonyl-2-{4-[1-(tert-butyldimethylsilyloxy)hexyl]phenyl}-5-oxo-cyclopentaneheptanoic acid, ethyl ester;
(d) removal of the methyl ester group at the C1-position and hydrolysis of the carbethoxy group with LiI in 2,4,6-collidine;
(e) removal of the tert-butyldimethylsilyl hydroxyl-protecting group.

A tenth aspect of the invention provides a method of making a compound comprising the following steps:
(a) asymmetric addition of (S)-1-(4-bromophenyl)-1-(tert-butyldimethylsilyloxy)hexane to 5-oxo-cyclopentenecarboxylic acid, (1R,2S,3R,4S)-{3-[N-benzenesulfonyl-N-(3,5-dimethylphenyl)amino]-2-bornyl}ester in the presence of an organo-copper agent and an organolithium agent to give (1R,2S)-2-{4-[1-(S)-(tert-butyldimethylsilyloxy)hexyl]phenyl}-5-oxo-cyclopentanecarboxylic acid, (1R,2S,3R,4S)-{3-[N-benzenesulfonyl-N-(3,5-dimethylphenyl)amino]-2-bornyl}ester;
(b) conversion of the 3-[N-benzenesulphonyl-N-(3,5-dimethylphenyl)-amino]-2-bornyl group to a methyl group by reaction with methanol to give (1R,2S)-2-{4-[1-(S)-(tert-butyldimethylsilyloxy)hexyl]phenyl}-5-oxo-cyclopentanecarboxylic acid, methyl ester;
(c) treating the (1R,2S)-2-{4-[1-(S)-(tert-butyldimethylsilyloxy)hexyl]phenyl}-5-oxo-cyclopentanecarboxylic acid, methyl ester with ethyl-7-bromoheptanoate in the presence of base to give (2S)-1-methoxycarbonyl-2-{4-[1-(S)-(tert-butyldimethylsilyloxy)hexyl]phenyl}-5-oxo-cyclopentaneheptanoic acid, ethyl ester;
(d) removal of the methyl ester group at the C1-position and hydrolysis of the (6-carboethoxy)hexyl ester group, with LiI in 2,4,6-collidine giving (1R,2S)-2-{4-[1-(S)-(tert-butyldimethylsilyloxy)hexyl]phenyl}-5-oxo-cyclopentaneheptanoic acid;
(e) removal of the tert-butyldimethylsilyloxy hydroxyl-protecting group.

An eleventh aspect of the present invention provides a compound obtainable by or obtained by the methods of any one of the seventh to tenth aspects. A twelfth aspect of the invention provides a method of making a compound according to any one of the the first to sixth aspects of the invention, comprising one or more steps as described in the general synthesis section below.

A further aspect of the present invention provides a compound of any one of the first to sixth aspects, or a compound made (or obtainable) by the methods of any one of the seventh to tenth or twelfth aspects, or a pharmaceutically acceptable salt thereof for use in a method of therapy.

Another aspect of the present invention provides a pharmaceutical composition comprising a compound of any one of the first to sixth aspects, or a compound made by the methods of any one of the seventh to tenth or twelfth aspects, or a pharmaceutically acceptable salt thereof together with a pharmaceutically acceptable carrier or diluent.

A further aspect of the present invention provides the use of a compound of any one of the first to sixth aspects, or a compound made by (or obtainable by) the methods of any one of the the seventh to tenth or twelfth aspects, or a pharmaceutically acceptable salt thereof in the preparation of a medicament for the treatment of a condition alleviated by agonism of an EP₂ receptor.

Other aspects of the present invention provide methods of synthesizing the compounds of the invention, or relevant intermediates, by the methods set out below.

Another aspect of the present invention provides a method of treating a condition which can be alleviated by agonism of an EP₂ receptor, which method comprises administering to a patient in need of treatment an effective amount of a compound of any one of the first to sixth aspects, or a compound made by (or obtainable by) the methods of any one of the seventh to tenth or twelfth aspects, or a pharmaceutically acceptable salt thereof.

Conditions which can be treated by agonism of an EP₂ receptor are discussed above, and particularly include dysmenorrhoea, pre-term labour, glaucoma, osteoporosis, asthma, allergy, bone disease, fracture repair, infertility, male sexual dysfunction, female sexual dysfunction, periodontal disease, gastric ulcer and renal disease.

EP receptor agonists are known to be able to inhibit T-cell activation and the release of pro-inflammatory cytokines, although the EP receptor involved in mediating these effects in human T-cells has not been previously defined. The present inventors have discovered that EP₂ agonists inhibit human T-cell activation (proliferation) and inhibit the release of multiple pro-inflammatory cytokines including interleukin 2 (IL-2) tumour necrosis factor (TNF_{α}) and interferon gamma (IFNγ). This profile of activity strongly suggests that EP₂ receptor agonists will be useful in treating immune and inflammatory disorders, including but not limited to psoriasis, psoriatic arthritis, dermatitis, rheumatoid arthritis, transplant rejection, inflammatory bowel disease, systemic lupus erythematosus, graves disease, scleroderma, multiple sclerosis, Type I diabetes, and transplant rejection, and in particular psoriasis (Griffiths, C., Current Drugs Targets—Inflammation & Allergy, 3, 157-161, (2004); Lebwohl, M., Lancet, 361, 1197-1204 (2003); Salim, A. & Emerson, R., Curr. Opin. Investig. Drugs, 2(11), 1546-8 (2001)). Therefore, a further condition which can be alleviated by agonism of an EP₂ receptor is psoriasis.

Furthermore, the present inventors have also shown that EP₂ receptor agonists inhibit the release of the pro-inflammatory cytokine, TNF_{α} from human monocytes and alveolar macrophages. This profile of activity adds further evidence to the view that that EP₂ receptor agonists will be useful in treating immune and inflammatory disorders and in particular, inflammatory lung diseases (including, but not limited to: asthma, chronic obstructive pulmonary disease, acute respiratory distress syndrome, pulmonary fibrosis and cystic fibrosis)).

Furthermore, aspects of the present invention relate to the use of EP₂ agonists to treat conditions ameliorated by the inhibition of IL-2 TNF_{α} and/or IFNγ production and the use of an EP₂ agonist in the preparation of a medicament for the treatment of a condition alleviated by inhibition of IL-2 production.

The present invention also provides methods of stimulating EP₂ receptors and/or inhibiting the production of IL-2, TNF_{α} and/or IFNγ, in vitro or in vivo, comprising contacting a cell with an effective amount of a compound of the first to third aspects, or a compound made (or obtainable) by the methods of the fourth, fifth, sixth, seventh or ninth aspects.

In some embodiments, the compounds described above may show selectivity for EP₂ receptors relative to the other three EP receptors, i.e. EP₁, EP₃ and EP₄. This selectivity allows for targeting of the effect of the compounds of the invention, with possible benefits in the treatment of certain conditions.

The invention will be described with reference to the attached figures, in which:

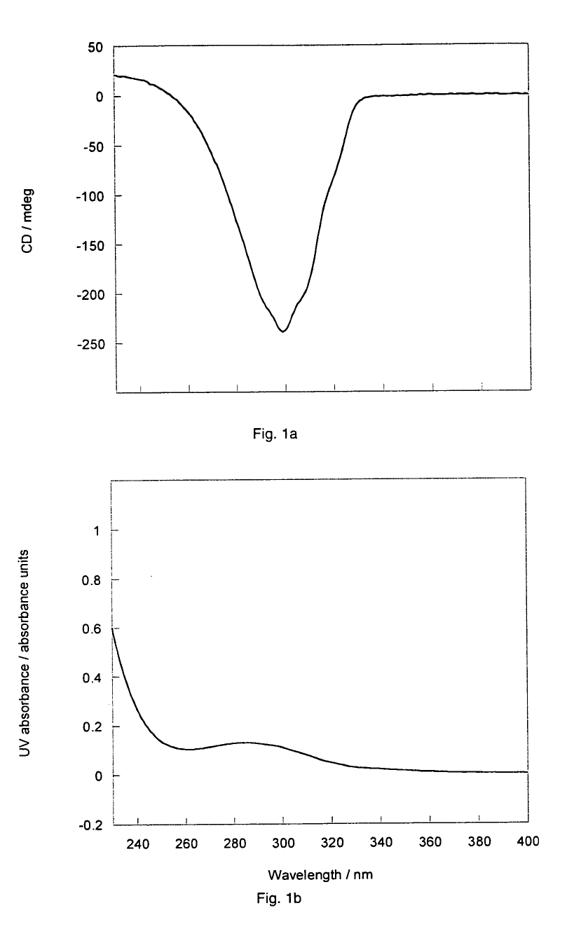

FIG. 1a shows the CD spectrum of prostaglandin E₂ (PGE₂) (0.7 mg/mL) in ethanol using a 1.0 cm pathlength cuvette.

FIG. 1b shows the UV spectrum of PGE₂ (0.7 mg/mL) in ethanol using a 1.0 cm pathlength cuvette.

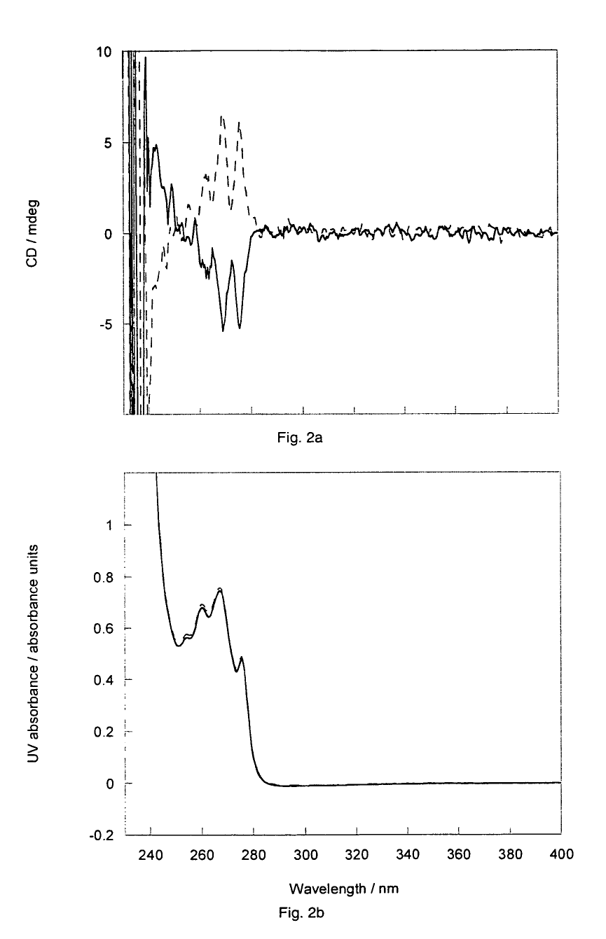

FIG. 2a shows the CD spectrum of (R)-1-(4-bromophenyl)hexan-1-ol (R-BPH) (solid line in figure) (0.7 mg/mL) and (S)-1-(4-bromophenyl)hexan-1-ol (S-BPH) (dashed line in figure) (0.7 mg/mL) in ethanol using a 1.0 cm pathlength cuvette.

FIG. 2b shows the UV spectrum of R-BPH (solid line in figure) (0.7 mg/mL) and S-BPH (dashed line in figure) (0.7 mg/mL) in ethanol using a 1.0 cm pathlength cuvette (N.b. solid and dashed lines almost overlie each other in this figure).

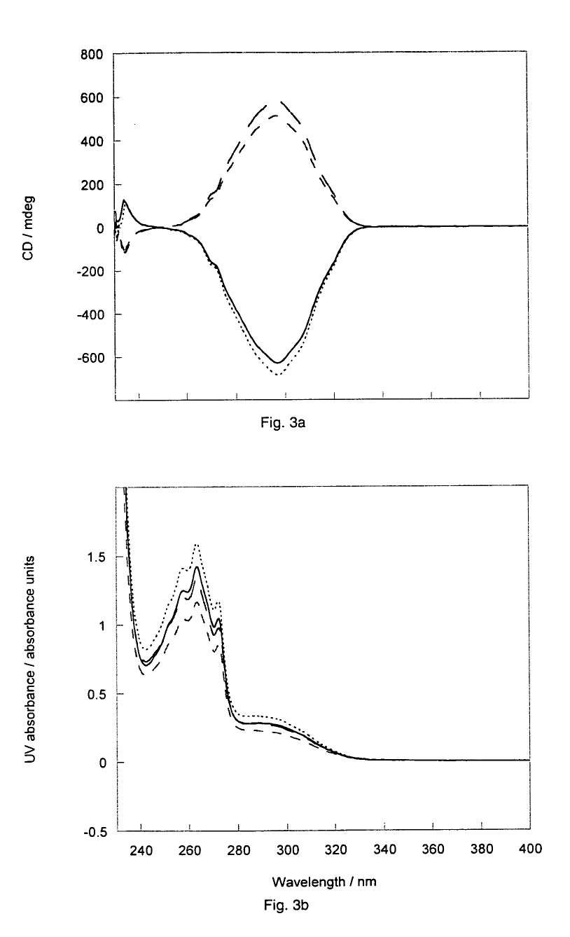

FIG. 3a shows the CD spectrum of each of the four trans-stereoisomers of Example 4 (compounds A, C, E and G) (all 19 mg/mL) in ethanol using a 0.1 cm pathlength cuvette.

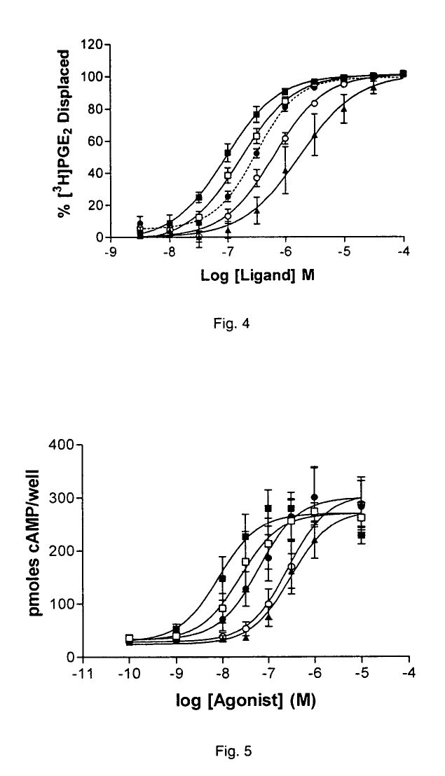

FIG. 4 shows the variation in percentage of [³H]PGE₂ displaced with concentration of five test compounds in an assay of binding ability to human EP₂ receptors;

FIG. 5 shows the variation in concentration of cAMP following stimulation by five test compounds in an assay of human EP₂ receptor stimulation;

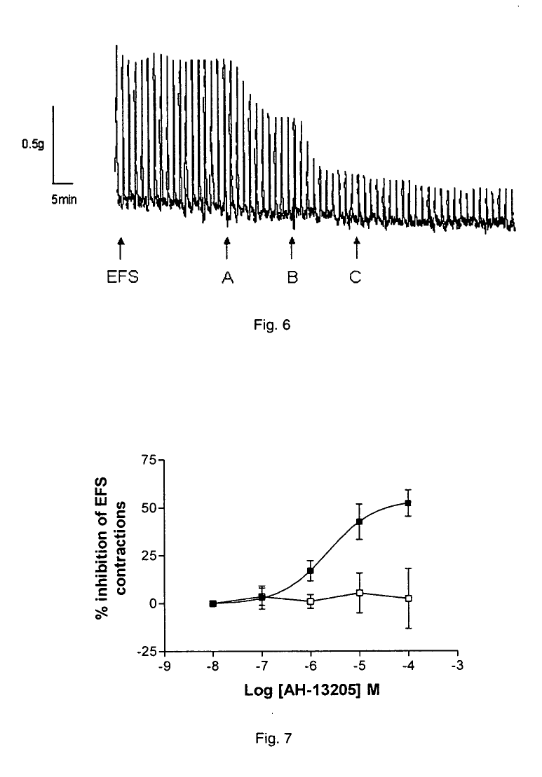

FIG. 6 shows the effect on human myometrial activity of AH13205;

FIG. 7 shows the variation in % inhibition of electrical field stimulation (EFS) induced contractions with concentrations of AH13205 and delivery vehicle or delivery vehicle alone in an assay of human myometrial activity;

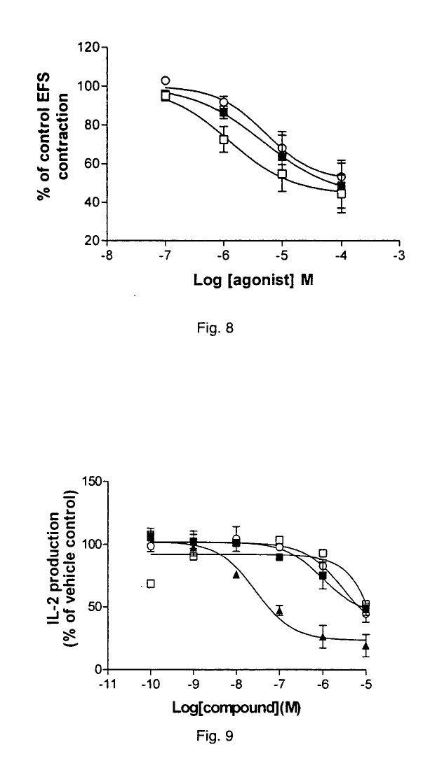

FIG. 8 shows the variation in % of control electrical field stimulation (EFS) induced contractions with concentrations of three test compounds in an assay of human myometrial activity;

FIG. 9 shows the variation in IL-2 production with concentration of 4 test compounds in a lymphocyte assay;

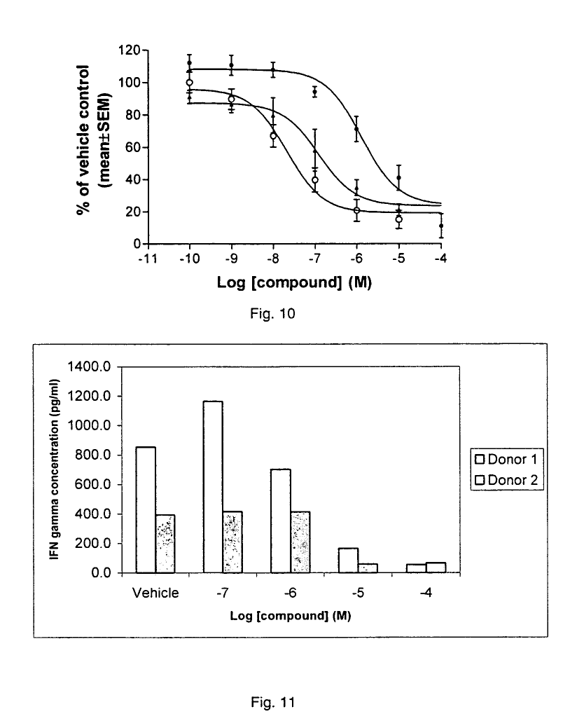

FIG. 10 shows the variation of IL-2 production with concentration of 3 EP₂ receptor agonists in a lymphocyte assay;

FIG. 11 shows the variation of Interferon gamma release with concentration of 3 EP₂ receptor agonists in a lymphocyte assay;

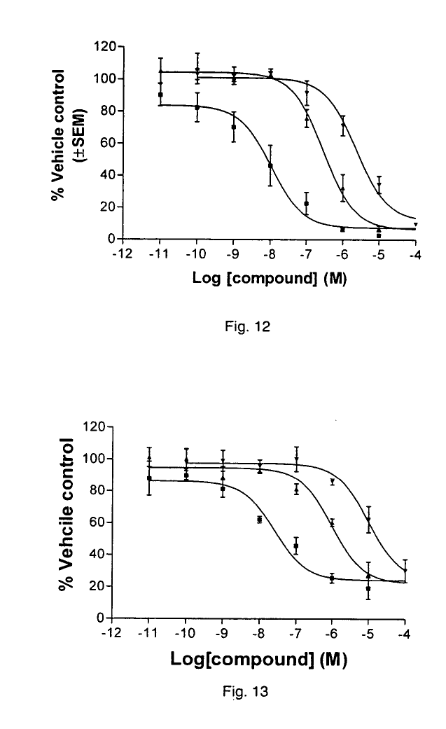

FIG. 12 shows the variation of TNFα production in response to 3 EP₂ receptor agonists in a lymphocyte assay;

FIG. 13 shows the variation of cell proliferation in response to 3 EP₂ receptor agonists in a lymphocyte assay;

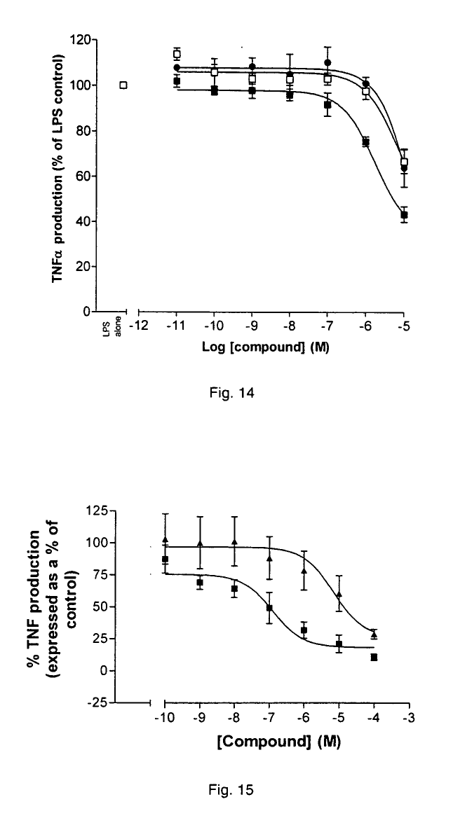

FIG. 14 shows the variation of TNFα production in response to 3 test compounds in a monocyte assay;

FIG. 15 shows the variation of TNFα production in response to 2 test compounds in an alveolar macrophage assay.

### DEFINITIONS

Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to a hydroxyl group also includes the anionic form (—O⁻), a salt or solvate thereof, as well as conventional protected forms of a hydroxyl group.

Salts, Solvates and Protected Forms

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g. pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

A wide variety of such “protecting”, “blocking”, or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, a carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

Prodrugs

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also include prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:
C₁₋₇ alkyl
(e.g., -Me, -Et, -nPr, -iPr, -nBu, -sBu, -iBu, -tBu);
C₁₋₇ aminoalkyl
(e.g., aminoethyl; 2-(N,N-diethylamino)ethyl;
2-(4-morpholino)ethyl);
C₁₋₇ hydroxy or polyhydroxt alkyl
(e.g. 2-hydroxyethyl, 2.3-dihydroxypropyl (glyceryl)) and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Treatment and Therapy

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “therapeutically-effective amount”, as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. Suitable dose ranges will typically be in the range of from 0.01 to 20 mg/kg/day, preferably from 0.1 to 10 mg/kg/day, although the dose may be as low as from about 0.00001 to 1 mg/day in the case of the topical ocular administration.

Compositions and Their Administration

Compositions may be formulated for any suitable route and means of administration. Pharmaceutically acceptable carriers or diluents include those used in formulations suitable for oral, rectal, nasal, topical (including buccal, ocular and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, polyoxol esters and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, 20th edition, pub. Lippincott, Williams & Wilkins, 2000. The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

Formulations suitable for transdermal administration include gels, pastes, ointments, creams, lotions, and oils, as well as patches, adhesive plasters, bandages, dressings, depots, and reservoirs.

Ointments are typically prepared from the active compound and a paraffinic or a water-miscible ointment base.

Creams are typically prepared from the active compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues. Formulations suitable for vaginal administration may be presented as pessaries, tampons, creams, gels, pastes, foams or spray formulations containing in addition to the active compound, such carriers as are known in the art to be appropriate.

Eye drops, for topical ocular administration preferably comprise between 0.001 and 20% of the active agent with the remainder made up from well known carriers such as water or saline, or other additives as discussed below.

Typical ocular compositions may include:
(a) antimicrobial preservatives—suitable preservatives include: benzalkonium chloride, thimerosal, chlorobutanol, methyl paraben, propyl paraben, phenylethyl alcohol, edetate disodium, sorbic acid, onamer, or other agents known to those skilled in the art. Such preservatives are typically employed at a concentration between about 0.001% and about 1.0% by weight;
(b) solubilising agents, such as Polysorbate 20, 60 and 80; Pluronic F-68, F-84 and P-103; Tyloxapol; Cremophor; sodium dodecyl sulfate; glycerol; PEG 400; propylene glycol; cyclodextrins; or other agents known to those skilled in the art. Such co-solvents are typically employed at a concentration between about 0.01% and about 2% by weight; and
(c) viscosity agents, such as polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art. Such agents are typically employed at a concentration between about 0.01% and about 2% by weight.

Other optional components, sometimes termed ‘inactive ingredients’, include sodium chloride, sodium dihydrogen phosphate monohydrate and/or anhydrous, polyoxyl 40 hydrogenated caster oil, tromethamine, boric acid, mannitol, edetate disodium, sodium hydroxide and/or hydrochloric acid to adjust pH and purified water.

Ocular formulations containing prostaglandins are described in, amongst others: U.S. Pat. No. 5,889,052; U.S. Pat. No. 4,599,353; U.S. Pat. No. 6,011,062; U.S. Pat. No. 6,235,781; U.S. Pat. No. 5,849,792; U.S. Pat. No. 5,631,287, which are herein incorporated by reference.

General Synthesis Methods

Compounds of formula 1: 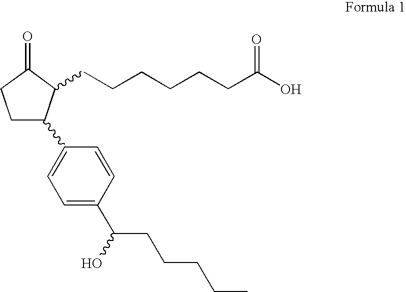
wherein 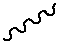
represents a defined stereochemistry at each chiral centre, and the groups on the pentanone are trans to one another, may be synthesised from compounds of formula 2: 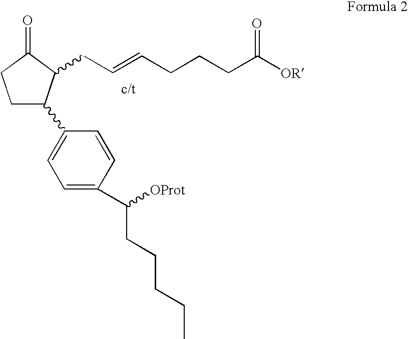
having the same stereochemistry at each chiral centre, wherein R′ represents a C₁₋₇ alkyl group (a monovalent moiety obtained by removing a hydrogen atom from a carbon atom of a hydrocarbon compound having from 1 to 7 carbon atoms, e.g. methyl (C₁), ethyl (C₂), propyl (C₃), butyl (C₄), pentyl (C₅), hexyl (C₆), heptyl (C₇)) by reduction of the double bond and deprotection of the acid and alcohol using standard techniques e.g. the reduction may be carried out with hydrogen, palladium on charcoal in a solvent such as ethyl acetate at normal temperature and pressure. A particularly preferred alcohol protecting group is a silyl group, such as tert-butyldimethylsilyl (TBDMS), which can be removed, for example, with aqueous acid and a co-solvent, which conditions may also deprotect the acid group. These reactions may be carried out in either order. The double bond may be either in the cis- or trans- orientation, or a mixture of these.

Compounds of formula 2 may be synthesised by trapping an enolate of formula 3: 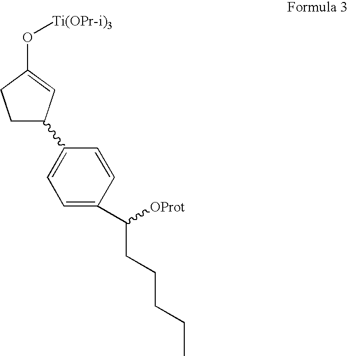
having the same stereochemistry at each of the two chiral centres, with a compound of formula 4: 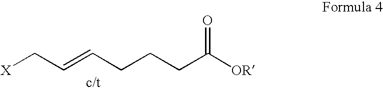
wherein X is a leaving group, such as halide or mesylate, and R′ is as in formula 2, in the presence of a strong base, such as Li Oi-Pr at room temperature.

The four enolates of formula 3 may be generated from cyclopent-2-enone (formula 5): 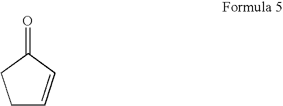
by reacting it with a compound of formula 6: 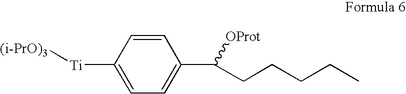
having the same stereochemistry at the chiral centre, in the presence of a transition metal catalyst, preferably Rh(I), in the presence of a chiral ligand, such as BINAP, (2,2′-Bis(diphenylphosphino)-1,1′-binaphthyl). Using S-BINAP would yield enolates of formula 3a: 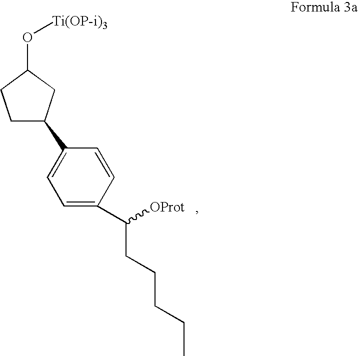
whereas using R-BINAP would yield enolates of formula 3b:

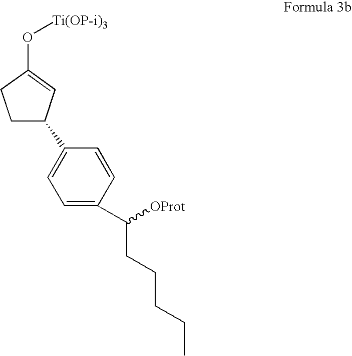

The compounds of formula 6 may be generated in situ from the reaction of compounds of formula 7: 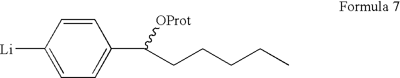
having the same stereochemistry at the chiral centre, with ClTi(Oi-Pr)₃, before the reaction of compounds of formulae 5 and 6. This reaction may be carried generally in accordance with the methods described in Hayashi, T., et al., JACS, 124, 12102-12103 (2002), such as 1.6 equivalents of the compound of formula 6 to the compound of formula 5, with 3% of the catalyst in tetrahydrofuran at 20° C. for 1 hour under an inert atmosphere.

Compounds of formula 7 can be generated from the corresponding bromo compound of formula 8: 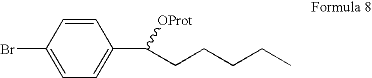
with the same stereochemistry at the chiral centre, by treating with an alkyl lithium, in a solvent, for example THF.

Compounds of formula 8 are made by protecting compounds of formula 9: 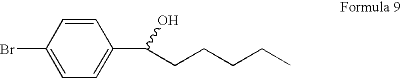
with the same stereochemistry at the chiral centre, using standard conditions, which retain the stereochemistry of the chiral centre, e.g. reaction with TBDMSCl.

The single stereoisomers of compound 9 can by made from a compound of formula 10: 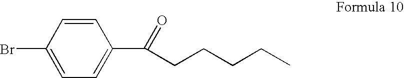
by either enantioselective reduction (e.g. see Brown, H. C., et al. J. Am. Chem. Soc., 110, 1539-1546 (1988)), or by reduction to the racemate of compound 9 followed by optical resolution.

Compounds of formula 4 are known from the synthesis of natural prostaglandins, e.g. Suzuki, M., et al., J. Am. Chem. Soc., 107, 3348-3349 (1985), and may be synthesised by a variety of routes.

One route, based on a route disclosed in Taber, D. F., et al., J. Org. Chem., 62, 194-198 (1997), is as follows:

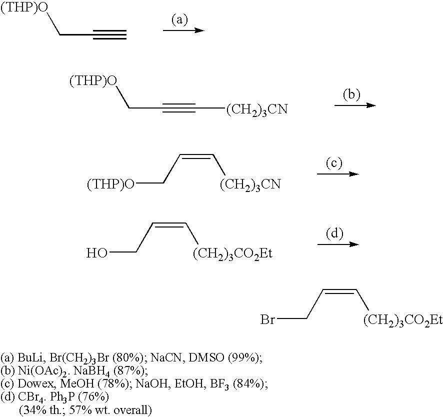

Other possible methods use different alkylating agents for the propargyl alcohol (and are illustrated below), but preparation of the alkylating agents require additional step. An example of this is alkylation of the ortho ester of bromobutyrate (Patterson, J. W., et al., Synthesis, 1985, 337-338). The ortho ester of 5-hexynoic acid has been reacted with BuLi/formaldehyde to give the same intermediate (Syn. Comms. 1989, p. 1509). A further possible route may involve the direct reaction of 5-hexynoic acid (commercially available, Aldrich) with base and formaldehyde.

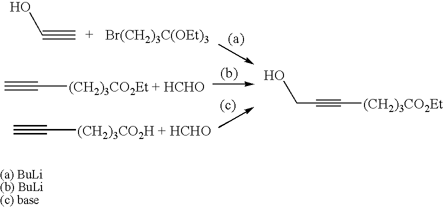

An alternative route to the four compounds of formula 1 is from compounds of formula 11: 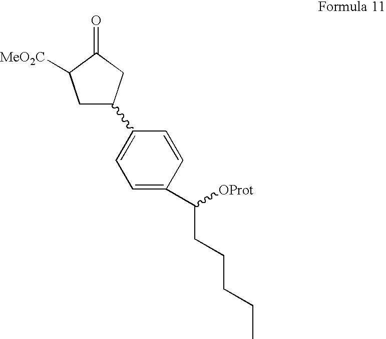
where the chiral centres have the same stereochemistry, by reaction first with sodium hydride and then with strong base, such as potassium amide or butyl lithium, to form the dianion, (Weiler, L., J. Am. Chem. Soc., 92, 6702-6704 (1970) and see, for example, Modern Synthetic Reactions, 2^{nd} Edition 1972, H.O. House, p. 553), which can then be reacted with haloheptanoate to give the substituted ketoester (Huckin, S. N. and Weiler, L., Can. J. Chem., 52, 2157 (1974)), which subsequently can be hydrolysed and decarboxylated using standard conditions, e.g. treating with lithium iodide in collidine or treating with sodium cyanide in DMSO (see Modern Synthetic Reactions, 2^{nd} Edition 1972, H.O. House, p. 511-517).

It may be necessary to replace the haloheptanoate with a more reactive alkylating agent such as the allylic halide of Formula 4, followed by reduction of the double bond.

The compounds of formula 11 can be synthesised from compounds of formula 12: 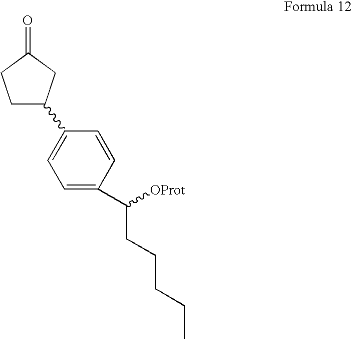
having the same stereochemistry at each of the two chiral centres, by reaction with dimethyl carbonate and a base, such as sodium hydride, in a solvent such as toluene or THF, with mild heating.

The compounds of formula 12 can be synthesised from cyclopent-2-enone (formula 5): 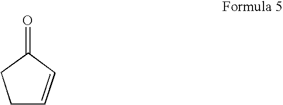
by boronic acid addition (see Takaya, Y., et al., J. Am. Chem. Soc., 120, 5579-5580 (1998) and Hayashi, T., Synlett, SI, 879-887 (2001)) of a compound of formula 13: 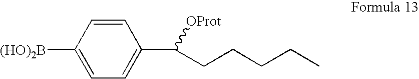
with the same stereochemistry at the chiral centre, in the presence of a transition metal catalyst, preferably Rh(I), in the presence of a chiral ligand, preferably BINAP. Suitable conditions include the use of 3% catalyst and chiral ligand in aqueous dioxane at 60° C. for 20 hours.

By analogy with established chemical precedent, use of S-BINAP yields compounds of formula 12a: 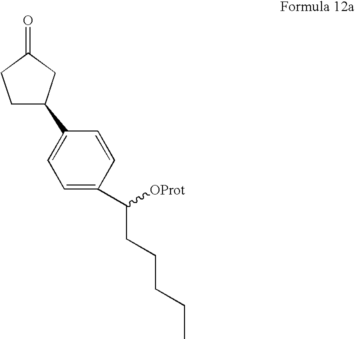
whilst using R-BINAP yields compounds of formula 12b:

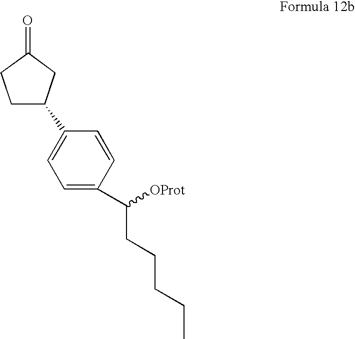

Compounds of formula 13 may be generated from compounds of formula 8, with the same stereochemistry at the chiral centre, by standard techniques. Such techniques include first treatment with a lithium exchange reagent, for example t-butyl lithium, in a solvent, for example THF, at a suitable temperature (for butyl lithium in THF, −78° C.). This is followed by treatment with an appropriate boron reagent, for example B(Oⁱ⁻Pr)₃ followed by hydrolysis, e.g. by potassium hydroxide (Thompson, W. J. and Gaudino, J., J. Org. Chem., 49, 5237-5243 (1984)).

An alternative route from compounds of formula 13 to compounds of formula 11 where the chiral centres have the same stereochemistry is reaction of compounds of formula 13 with the methylcarboxy substituted cyclopent-2-enone of formula 14: 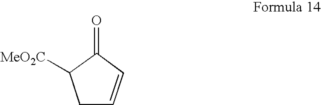
by boronic acid addition, in the presence of a transition metal catalyst, preferably Rh(I), in the presence of a chiral ligand, preferably BINAP. Suitable conditions include the use of 3% catalyst and chiral ligand in aqueous dioxane at 60° C. for 20 hours, i.e. similar reaction conditions used for the coupling of compound 5 with compounds of formula 13.

A further alternative route to the four compounds of formula 1 is from compounds of formula 15: 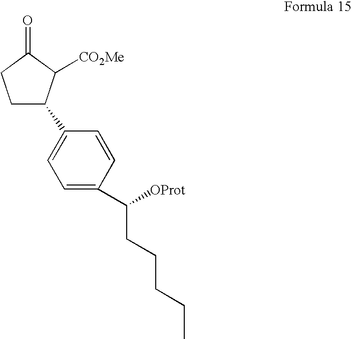
where the chiral centres have the same stereochemistry, by reaction with strong base, such as sodium hydride, e.g. in DMF, to form a monoanion, which can then be reacted with haloheptanoate to give the substituted ketoester, which subsequently can be hydrolysed and decarboxylated using standard conditions, e.g. treating with lithium iodide in collidine or treating with sodium cyanide in DMSO (see Modern Synthetic Reactions, 2^{nd} Edition 1972, H.O. House, p. 511-517). The trans arrangement on the cyclopentanone arises due to steric hindrance.

The compounds of formula 15 can be synthesized by coupling compounds of formula 13, with the same stereochemistry at the chiral centre, with the methylcarboxy substituted cyclopent-2-enone of formula 16: 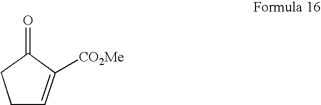
(Funk, R. L., et al., J. Am. Chem. Soc, 115, 8849-8850 (1993)), in the presence of a transition metal catalyst, preferably Rh(I), in the presence of a chiral ligand, preferably BINAP.

Suitable conditions include the use of 3% catalyst and chiral ligand in aqueous dioxane at 60° C. for 20 hours, i.e. similar reaction conditions used for the coupling of compound 5 with compounds of formula 13.

Alternatively, the stereoselective synthesis of (1R,2S)-2-[4-(1-(S)-hydroxyhexyl)phenyl]-5-oxo-cyclopentaneheptanoic acid (formula 17) may be achieved by the following general synthesis route.

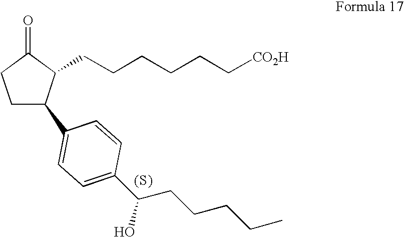

A compound of formula 17 may be formed from compound 18 in which the —OH group on the 4-hexyl-phenyl side chain is protected by an alcohol-protecting group.

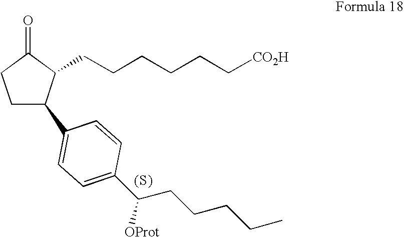

As mentioned previously, a particularly preferred alcohol-protecting group is a silyl group, such as tert-butyldimethylsilyl (TBDMS), which can be removed, for example, with aqueous acid and a co-solvent, for example THF.

Compounds of formula 18 may be formed from compounds of formula 19 by removal of the carboxymethyl group and hydrolysis of the heptanoate ester. This may be achieved, for example, by reaction with lithium iodide and 2,4,6-trimethylpyridine (collidine).

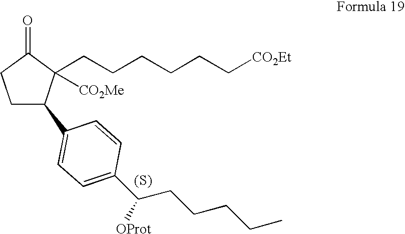

Compound 19 can be synthesised by addition of the heptyl-ethyl ester side chain to a compound of formula 20.

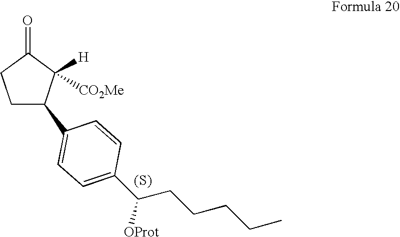

This can be achieved by the treatment of a compound of formula 20 with a suitable base in order to remove the hydrogen atom adjacent to the —CO₂Me group. The preferred base for this reaction is NaH in anhydrous solvent, such as DME.

Subsequent addition of ethyl heptanoate, activated at the 7-position, to the resultant compound gives a compound of formula 19. Preferably, the ethyl heptanoate is activated with a halogen atom in the 7-position. More preferably, this halogen atom is a bromine atom. It may also be necessary to incude a catalyst during the addition of the activated ethyl heptanoate. Such a catalyst is preferably sodium iodide.

Compounds of formula 20 maybe formed from compounds of formula 21 via a transesterification reaction.

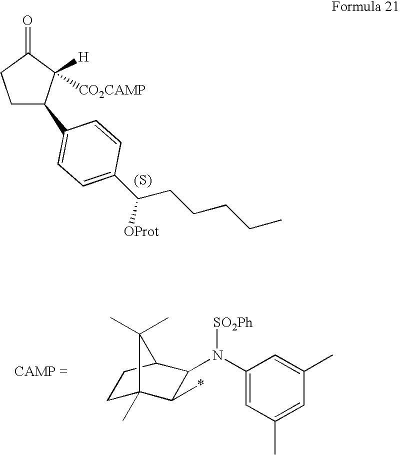

Formation of the methyl ester of compound 20 may be achieved by heating compound 21 with methanol in a sealed vessel.

Compounds of formula 21 can be formed by 1,4-addition to an unsaturated carbonyl compound of formula 22.

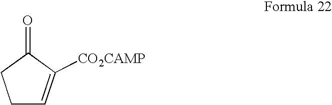

This can be achieved by reaction of an organometallic reagent of formula 23 with a copper (I) compound followed by addition to a compound of formula 22.

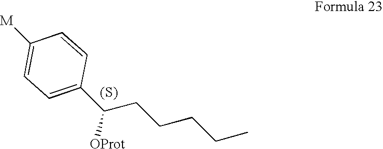

Where M in formula 23 represents an element which is less electronegative than copper. Preferably M is Li, MgX, BR₂ and ZnX, where X is a halogen atom.

The copper (I) reagent used in the coupling reaction of compounds of formulae 22 and 23 is preferably an anionic cuprate and is more preferably LiCu-(2-Th)CN (known as lithium 2-thienylcyanocuprate).

A compound of formula 23 may be formed by reaction of a halide of formula 24 by standard organometallic formation reactions.

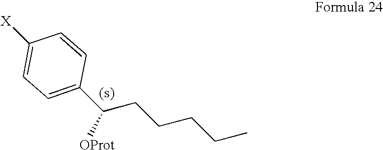

In formula 24, X is a halogen atom and is preferably I or Br.

Compounds of formula 22 may be formed from readily available starting materials by the method described in Tetrahedron 1996, 52, 971-986.

Acronyms

For convenience, many chemical moieties are represented using well known abbreviations, including but not limited to, methyl (Me), ethyl (Et), n-propyl (nPr), iso-propyl (iPr), n-butyl (nBu), sec-butyl (sBu), iso-butyl (iBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), tetrahydropyranyl (THP) and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), acetonitrile (ACN), trifluoroacetic acid (TFA), dimethylformamide (DMF), tetrahydrofuran (THF), ethyl acetate (EA), 1.2-dimehoxyethane (DME) and dimethylsulfoxide (DMSO).

Selectivity

The selectivity of the compound for agonising EP₂ receptors over the other EP receptors (i.e. EP₁, EP₃, EP₄) can be quantified by dividing the Ki for EP₂ (see below) by the Ki for the other EP receptors (see below). The resulting inverse ratio is preferably 10 or more, more preferably 100 or more.

### EXAMPLES

Nmr spectra were recorded on either a Bruker AV300 or Bruker DPX400. Mass spectra were recorded on a Waters ZMD Single Quadrapole Mass Spectrometer. Optical rotations were measured on a Perkin Elmer Polarimeter 341.

### Example 1

### Synthesis of Two Mixtures Each Containing 4 Stereoisomers of Methyl Esters of AH13205

### (a) (i) Synthesis of 1-(4-bromophenyl)hexan-1-one (1)

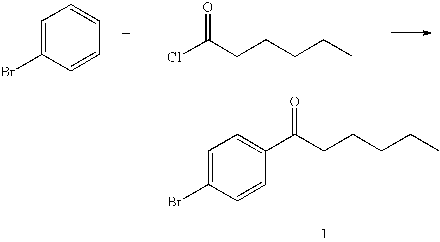

To a stirred ice-cooled mixture of AlCl₃ (83 g) and bromobenzene (200 mL) under nitrogen was added dropwise hexanoylchloride (75 mL) over a period of 30 minutes. The mixture was then heated to 80° C. (external) for 1.5 hours, after which time the solution had turned a deep red. The mixture was then allowed to cool before being poured into 600 mL ice water and then extracted with DCM (800 mL). The organic extracts were then washed with brine, dried (MgSO₄) and concentrated in vacuo. The concentrate was then treated with iso-hexane (1 L) and left in the freezer overnight, wherein crystallization took place. The slightly off-white solid was filtered and washed with more cold hexane, to yield the title compound (84 g). Shown to be adequately pure by nmr and tlc. ¹H NMR (CDCl₃, δ): 0.95 (3H, t); 1.4 (4H, m); 1.75 (2H, m); 2.95 (2H, t); 7.6 (2h, d); 7.85 (2H, d).

### (a) (ii) Synthesis of (S)-1-(4-Bromophenyl)hexan-1-ol (S-BPH) (2a)

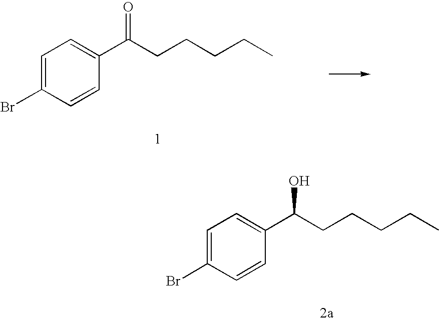

To a solution of (−)-DIP chloride [B-chlorodiisopinocampheylborane] (13.5 g) in anhydrous THF (20 ml), cooled to −25° C., was added a solution of 1-(4-bromophenyl)hexan-1-one (10 g) in anhydrous THF (20 ml) over 5-10 minutes keeping the temperature below −20° C. The mixture was kept at −25° C. for the next 6 hours then added to a vigorously stirred mixture of diethanolamine (12 ml) and triethylamine (10 ml) in ether (250 ml). The mixture was left stirring overnight, washed with dilute hydrochloric acid, brine, dried over sodium sulphate and evaporated in vacuo. Compound 2a (4.5 g; m.p. 70-71° C.) was obtained following silica-gel column chromatography of the residue in dichloromethane followed by re-crystallisation from heptane.

[α]_{D}²⁴=−26.5 (c=4.00; CHCl₃)

¹H NMR (CDCl₃, δ): 0.85 (3H, t); 1.2-1.9 (9H, m); 4.6 (1H, t); 7.15 (2H, d); 7.45 (2H, d).

HPLC (Chiracel OD 250×4.6 mm, eluant hexane:IPA 99:1, flow rate 0.5 ml/min, λ=254 nm): 44 minutes, e.e. 100%.

### (a) (iii) Synthesis of (R)-1-(4-Bromophenyl)hexan-1-ol (R-BPH) (2b)

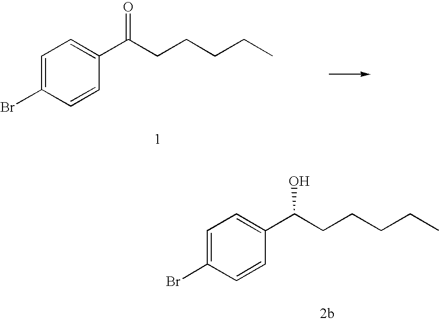

Compound 2b (4 g; m.p. 70-71° C.) was made from (+)-DIP chloride [B-chlorodiisopinocampheylborane] (13.5 g) and 1-(4-bromophenyl)hexan-1-one (10 g) by an analogous method to that described in Example 1(a)(ii).

[α]_{D}²⁴=+27.3 (c=4.06; CHCl₃)

m/z (EIMS): 256, 258

¹H NMR (CDCl₃, δ) 0.85 (3H, t); 1.2-1.9 (9H, m); 4.6 (1H, t); 7.15 (2H, d); 7.45 (2H, d).

HPLC (Chiracel OD 250×4.6 mm, eluant hexane: IPA 99:1, flow rate 0.5 ml/min, λ=254 nm): 47 minutes, e.e. 100%.

The absolute stereochemistry of compounds 2a and 2b was assigned by analogy with a literature method for reducing long chain aromatic ketones described by Brown, H. C., et al. J. Am. Chem. Soc., 110, 1539-1546 (1998). The alcohols were shown to be essentially homochiral by chiral HPLC.

### (a) (iv) Alternative synthesis of (S)-(−)-1-(4-bromphenyl)hexan-1-ol (S-BPH)(2a)

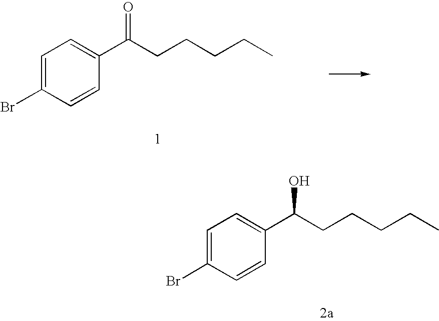

BH₃.THF (1 M in THF, 234 mL, 234 mmol) was stirred under nitrogen and cooled to −10° C. before being treated with (R)-2-methyl-CBS-oxazaborolidine (24 mL, 24 mmol). After being left stirring for 20 minutes, 1-(4-bromophenyl)hexan-1-one (1) (48.3 g) was added as a solution in THF (379 mL) over a period of 1 hour, and thereafter left for a further 20 minutes before quenching carefully with MeOH (100 mL)—H₂ evolves. Advisable to perform the quench at RT since MeOH reacts slowly at −10° C. The mixture was then concentrated in vacuo, then redissolved in MeOH (300 mL) and treated with HCl (2 M in Et₂O, 40 mL). The solution was stirred for 5 minutes before concentrating in vacuo, triturating with Et₂O and removing the solid by filtration. The mother liquors were again concentrated in vacuo then recrystallized from hexane (480 mL, 10 vol.) at −10° C. to yield the title compound as a fluffy white solid (20.7 g).

### (b) (i) Synthesis of (S)-1-(4-Bromophenyl)-1-(tert-butyldimethylsilyloxy)hexane (3a)

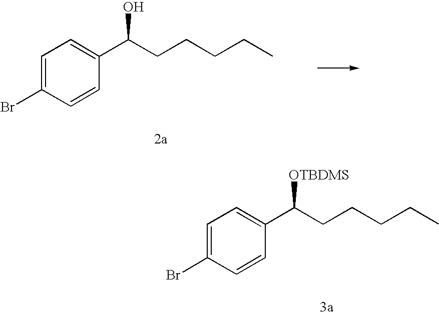

A mixture of S-BPH (2a)(10 g), tert-butyldimethylsilyl chloride (7 g) and imidazole (3.7 g) were stirred in anhydrous dimethylformamide (100 ml) for 16 hours. The mixture was partitioned between petroleum ether and water and the layers separated. The organic layer was washed with water, brine, dried over sodium sulphate and evaporated in vacuo. Compound 3a (14.5 g) was obtained as an oil following column chromatography of the residue in petroleum ether.

m/z (EIMS): 370, 372

¹H NMR (CDCl₃, δ): −0.2 (3H, s); 0.0 (3H, s); 0.85 (9H, s); 0.85 (3H, t); 1.25 (6H, m) 1.55 (2H, m); 4.6 (1H, m); 7.1 (2H, d); 7.4 (2H, d).

### (b)(ii) Synthesis of (R)-1-(4-Bromophenyl)-1-(tert-butyldimethylsilyloxy)hexane (3b)

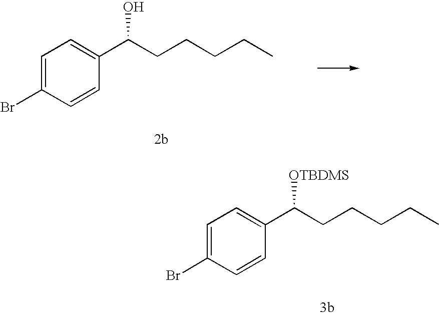

Compound 3b (18.5 g) was made from R-BPH (2b)(12.5 g) by an analogous method to that described in Example 1(b)(i).

m/z (EIMS): 370, 372

¹H NMR (CDCl₃, δ): −0.2 (3H, s); 0.0 (3H, s); 0.85 (9H, s); 0.85 (3H, t); 1.25 (6H, m) 1.55 (2H, m); 4.6 (1H, m); 7.1 (2H, d); 7.4 (2H, d).

### (c) Synthesis of 2-(6-carbomethoxyhexyl)cyclopent-2-en-1-one (5)

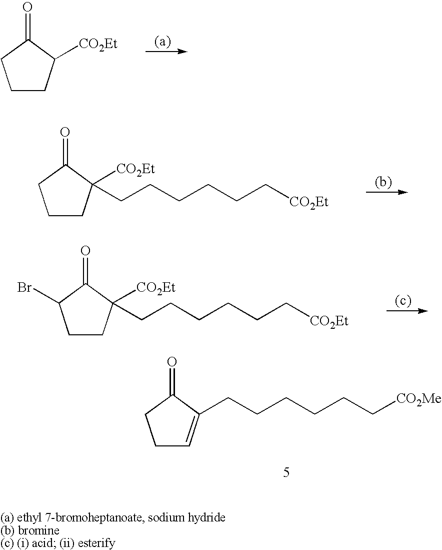

This known compound, which is commercially available, was prepared in three steps from ethyl 2-oxocyclopentane carboxylate by the methods of Bagli, J. et al., J. Org. Chem., 1972, 37, 2132-2138 and Bernady, K. F., J. Org. Chem., 1980 45, 4702-4715.

### (d) (i) Synthesis of 2-(4-[1-(R)-(tert-butyldimethylsilyloxy)hexyl]phenyl)-5-oxo-cyclopentaneheptanoic acid, methyl ester diastereomers (circa 3:1 trans: cis mixture)) (6b)

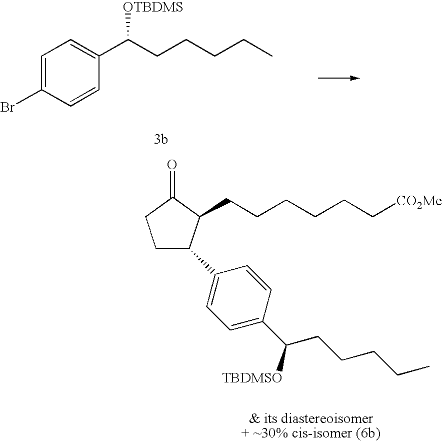

To a solution of (R)-1-(4-bromophenyl)-1-(tert-butyldimethylsilyloxy)hexane (3b) (7.2 g) in anhydrous diethyl ether (100 ml) was added tert-butyllithium (1.5M in hexanes; 28 ml) dropwise at −78° C., not allowing the temperature to rise above −60° C. The mixture was left at −78° C. for a further 3 hours. A slurry of copper (1) pentyne (2.5 g) in anhydrous diethyl ether (56 ml) was treated with hexamethylphosphorous triamide (8 ml) and the mixture stirred at room temperature for several minutes to form a solution. This freshly prepared solution was now added dropwise to the aryllithium solution at −78° C. and left for a further hour at −78° C., whereupon a solution of 2-(6-carbomethoxyhexyl)cyclo-pent-2-en-1-one (5) (4 g) in anhydrous diethyl ether (40 ml) was added. The reaction mixture was held at −78° C. for 15 minutes then at −25° C. to −10° C. for a further hour. The cold mixture was partitioned quickly between dilute hydrochloric acid and ether, the organic layer separated, washed with brine, dried over sodium sulphate and evaporated in vacuo. Compounds 6b (7.2 g) were obtained as a circa 3:1 mixture of trans:cis isomers following silica-gel column chromatography of the residue in 2:1 dichloromethane:petroleum ether then 3:17 ethyl acetate:petroleum ether.

¹H NMR (CDCl₃, δ)-trans-diastereomers: −0.25 (3H, s); 0.0 (3H, s); 0.85 (9H, s); 0.8-2.0 (22H, m); 2.2-2.6 (6H, m); 2.95 (1H, m); 3.65 (3H, s); 4.6 (1H, m); 7.15 (2H, d); 7.25 (2H, d).

¹H NMR (CDCl₃, δ)-cis-diastereomers: −0.27 (3H, s); −0.02 (3H, s); 0.85 (9H, s); 0.8-2.0 (22H, m); 2.2-2.6 (6H, m); 3.55 (1H, m); 3.65 (3H, s); 4.6 (1H, m); 7.0 (2H, d); 7.15 (2H, d).

### (d) (ii) 2-{4-[1-(S)-(tert-butyldimethylsilyloxy) hexyl]phenyl)-5-oxo-cyclopentaneheptanoic acid, methyl ester diastereomers (circa 3:1 trans:cis mixture) (6a)

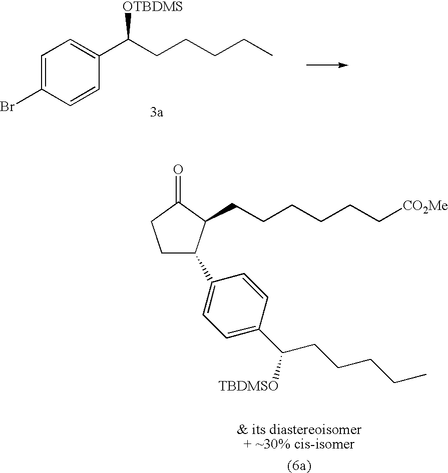

The title compound and its diastereoisomer, and about 30% of their cis-isomers (6a), (7.3 g) were made from (S)-1-(4-bromophenyl)-1-(tert-butyldimethylsilyloxy)hexane (3a) (7.2 g) and 2-(6-carbomethoxyhexyl)cyclopent-2-en-1-one (5) (4 g) by an analogous method to that described in Example 1(d)(i).

¹H NMR (CDCl₃, δ)-trans diastereomers: −0.25 (3H, s); 0.0 (3H, s); 0.85 (9H, s); 0.8-2.0 (22H, m); 2.2-2.6 (6H, m); 2.95 (1H, m); 3.65 (3H, s); 4.6 (1H, m); 7.15 (2H, d); 7.25 (2H, d).

¹H NMR (CDCl₃, δ)-cis diastereomers: −0.27 (3H, s); −0.02 (3H, s); 0.85 (9H, s); 0.8-2.0 (22H, m); 2.2-2.6 (6H, m); 3.55 (1H, m); 3.65 (3H, s); 4.6 (1H, m); 7.0 (2H, d); 7.15 (2H, d).

### (d)(iii) Alternative synthesis of 2-[4-[1-(R)-(tert-butyldimethylsilyloxy)hexyl]phenyl}-5-oxo-cyclopentaneheptanoic acid, methyl ester diastereomers (circa 3:1 trans: cis mixture) (6b)

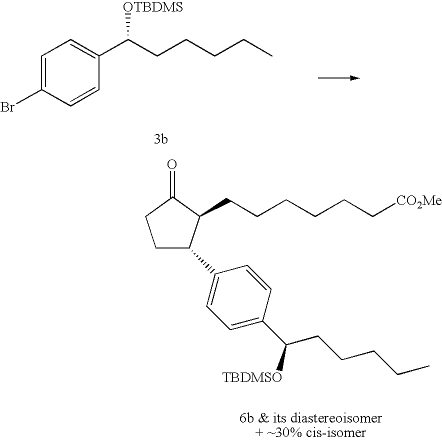

A mixture of (R)-1-(4-bromophenyl)-1-(tert-butyldimethylsilyloxy)hexane (3b)(0.8 g), magnesium turnings (0.11 g), a crystal of iodine and 1,2-dibromoethane (5 μl) in anhydrous THF (4 ml) were boiled to reflux to initiate reaction, then kept at 35° C. for 2 hours to form the Grignard solution. Lithium chloride (0.125 g) and copper (1) bromide.dimethyl sulphide complex (0.61 g) were stirred in anhydrous THF (4.5 ml) for a few minutes then cooled to −78° C. whereupon the Grignard solution was added dropwise. The resulting mixture was left for 5 minutes at −78° C. then trimethylsilyl chloride (0.38 ml) was added followed by a solution of 2-(6-carbomethoxyhexyl)cyclopent-2-en-1-one (0.18 g) in anhydrous THF (1.5 ml). The mixture was kept at −78° C. for 15 minutes, at 0° C. for 30 minutes then allowed to warm up to room temperature for an hour. The mixture was re-cooled to −20° C. whereupon dilute hydrochloric acid (4 ml) was added and the mixture stirred vigorously for two minutes. The cold mixture was partitioned between petroleum ether and saturated ammonium chloride solution and the layers separated. The organic layer was washed with brine, dried over sodium sulphate and evaporated in vacuo. Compounds 6b (0.20 g) were isolated as a mixture of cis and trans isomers following silica-gel column chromatography of the residue in 4:1 petroleum ether:ethyl acetate.

¹H NMR (CDCl₃, δ)-trans diastereomers: −0.25 (3H, s); 0.0 (3H, s); 0.85 (9H, s); 0.8-2.0 (22H, m); 2.2-2.6 (6H, m); 2.95 (1H, m); 3.65 (3H, s); 4.6 (1H, m); 7.15 (2H, d); 7.25 (2H, d).

¹H NMR (CDCl₃, δ)-cis diastereomers: −0.27 (3H, s); −0.02 (3H, s); 0.85 (9H, s); 0.8-2.0 (22H, m); 2.2-2.6 (6H, m); 3.55 (1H, m); 3.65 (3H, s); 4.6 (1H, m); 7.0 (2H, d); 7.15 (2H, d).

### (e)(i) Synthesis of trans and cis-2-[4-(1-(S)-hydroxyhexyl)phenyl]-5-oxo-cyclopentaneheptanoic acid, methyl ester diastereomers (7a) (Mixture 1)

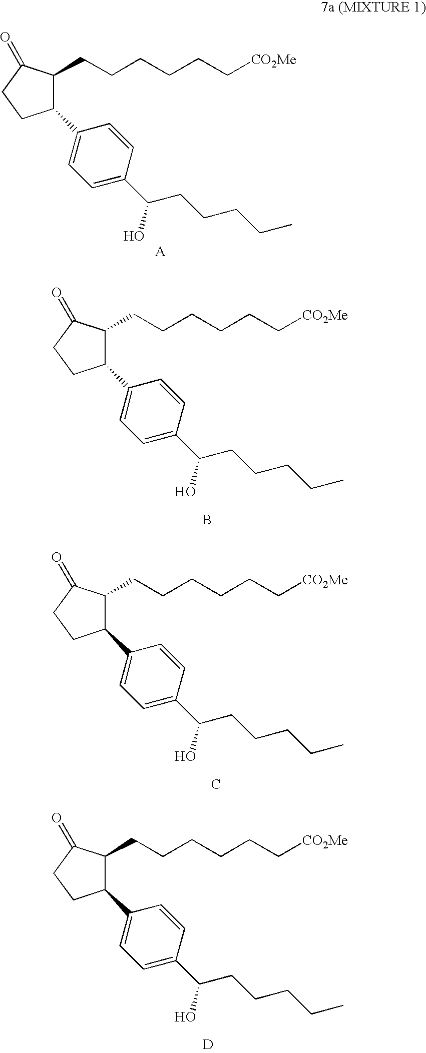 [0197] A: (1S,2R)-2-[4-(1-(S)-hydroxyhexyl)phenyl]-5-oxo-cyclopentaneheptanoic acid, methyl ester [SRS]
B: (1R,2R)-2-[4-(1-(S)-hydroxyhexyl)phenyl]-5-oxo-cyclopentaneheptanoic acid, methyl ester [RRS]
C: (1R,2S)-2-[4-(1-(S)-hydroxyhexyl)phenyl]-5-oxo-cyclopentaneheptanoic acid, methyl ester [RSS]
D: (1S,2S)-2-[4-(1-(S)-hydroxyhexyl)phenyl]-5-oxo-cyclopentaneheptanoic acid, methyl ester [SSS]

2-{4-[1-(S)-(tert-butyldimethylsilyloxy)hexyl]phenyl}-5-oxo-cyclopentaneheptanoic acid, methyl ester diastereomers (6a) (0.2 g; of circa 3:1 trans:cis composition) was stirred in a mixture of THF (3.5 ml) and dilute hydrochloric acid (2M; 1 ml) for 20 hours at 25° C. The reaction mixture was added to brine and extracted twice with dichloromethane. The combined organic layers were dried over sodium sulphate and evaporated in vacuo. 7a (Mixture 1) (0.095 g) was obtained as an oil (of circa 95:5 trans:cis composition) following silica-gel column chromatography of the residue in 3:1 petroleum ether:ethyl acetate then 200:3 dichloromethane:methanol.

m/z (EIMS): 402

¹H NMR (CDCl₃, δ)-trans diastereomers only: 0.8-2.0 (23H, m); 2.2-2.6 (6H, m); 2.95 (1H, m); 3.65 (3H, s); 4.65 (1H, t); 7.25 (2H, d); 7.35 (2H, d).

### (e) (ii) trans and cis-2-[4-(1-(R)-hydroxyhexyl)phenyl]-5-oxo-cyclopentaneheptanoic acid, methyl ester diastereomers (7b) (Mixture 2)

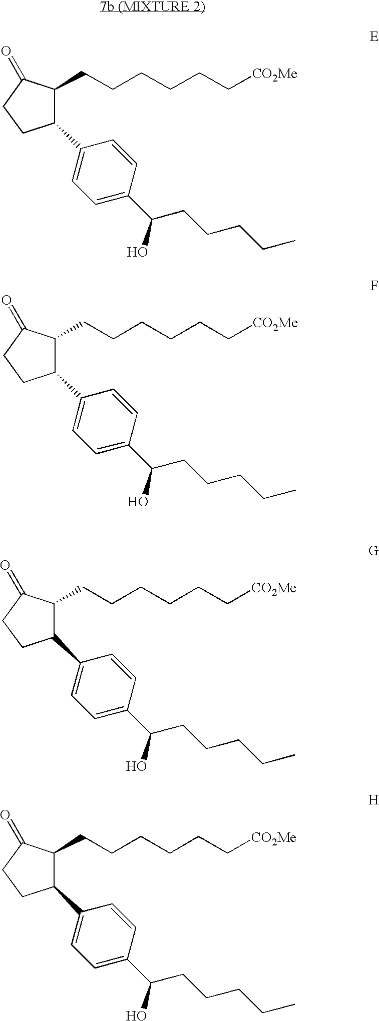 [0204] E: (1S,2R)-2-[4-(1-(R)-hydroxyhexyl)phenyl]-5-oxo-cyclopentaneheptanoic acid, methyl ester [SRR]
F: (1R,2R)-2-[4-(1-(R)-hydroxyhexyl)phenyl]-5-oxo-cyclopentaneheptanoic acid, methyl ester [RRR]
G: (1R,2S)-2-[4-(1-(R)-hydroxyhexyl)phenyl]-5-oxo-cyclopentaneheptanoic acid, methyl ester [RSR]
H: (1S,2S)-2-[4-(1-(R)-hydroxyhexyl)phenyl]-5-oxo-cyclopentaneheptanoic acid, methyl ester [SSR]

Compounds 7b (0.15 g; of circa 95:5 trans:cis composition)) were made from 2-{4-[1-(R)-(tert-butyldimethylsilyloxy)hexyl]phenyl}-5-oxo-cyclopentaneheptanoic acid, methyl ester diastereomers (0.3 g; of circa 3:1 trans:cis composition)(6b) by an analogous method to that described in Example 1(e)(i).

m/z (EIMS): 402

¹H NMR (CDCl₃, δ)-trans isomer only: 0.8-2.0 (23H, m); 2.2-2.6 (6H, m); 2.95 (1H, m); 3.65 (3H, s); 4.65 (1H, t); 7.25 (2H, d); 7.35 (2H, d).

### Example 2

### Separation of trans-2-[4-(1-hydroxyhexyl)phenyl]-5-oxo-cyclopentaneheptanoic acid methyl ester diastereomers

HPLC of a 90 mg sample of either ester mixture 1 or 2 on a chiral stationary phase (ChiralPak AD, Daicel Chemical Industries, Japan) using a mobile phase of 100% ethanol afforded complete separation on a column of 25 cm in length by 2 cm internal diameter, in about an hour. A 1 g sample of either mixture was separated in eleven consecutive 90 mg runs (flow rate 4 ml/min; detection 230 nm). The recovered esters were then hydrolysed to the acids as follows. Methyl ester (0.45 g) in 4:1 v/v tetrahydrofuran in water (40 ml) was treated with 1M lithium hydroxide in water (1.37 ml, 1.2 equiv.) added dropwise and the solution was stirred overnight at ambient temperature. The solution was concentrated in vacuo, diluted with water, acidified to pH˜1 and extracted into ethyl acetate. The extract was dried over magnesium sulphate, filtered and concentrated in vacuo at 30° C. to give the acid as an oil. The recoveries of acids starting from 1 g of each mixture of methyl esters were: from Peak 1, mixture 1: 0.392 g; from Peak 2, mixture 1: 0.427 g; from Peak 1, mixture 2: 0.433 g and from Peak 2, mixture 2: 0.381 g.

Optical rotations were recorded for the acids derived from each of the methyl ester peaks indicated. The acids obtained from the isolated peaks are hereinafter referred to as Peak m, Mixture m acid, for clarity.

Peak 1, mixture 1 acid

[α]²⁵ −422.4 (365 nm), −142.9 (436 nm), −57.3 (546 nm), −47.9 (578 nm), −45.1 (589 nm)
(c=0.6275, CHCl₃; path length 100 mm)
   Peak 2, mixture 1 acid
[α]²⁵ +314.9 (365 nm), +80.6 (436 nm), +22.7 (546 nm), +17.3 (578 nm), +16.0 (589 nm) (c=1.365, CHCl₃; path length 100 mm)
   Peak 1, mixture 2 acid
[α]²⁵ −299.4 (365 nm), −77.8 (436 nm), −22.2 (546 nm), −16.9 (578 nm), −15.5 (589 nm)
(c=0.81, CHCl₃; path length 100 mm)
   Peak 2, mixture 2 acid
[α]²⁵ +422.6 (365 nm), +140.6 (436 nm), +55.5 (546 nm), +45.9 (578 nm), +42.9 (589 nm)
(c=0.885, CHCl₃; path length 100 mm)

Chemical purity was determined by NMR and LC-MS; chiral purity was determined by chiral HPLC as described below. ¹H NMR (CDCl₃) confirmed the structure of the acid and showed the presence of only a trace of the methyl ester; a low level impurity, probably the cis-isomer, was always present together with residual ethyl acetate.

¹³C NMR showed very small differences between diastereomers of the esters. Peak 1, mixture 1 acid and peak 2, mixture 2 acid were shown to be enantiomers, as were peak 2, mixture 1 acid and peak 1, mixture 2 acid. This correlates with the optical rotation data for each compound shown above.

### Example 3

### Hydrolysis of Separated Methyl Esters

0.45 g of a methyl ester (as separated in Example 2) was dissolved in 40 ml of a 4:1 v/v solution of THF in water; 1.37 ml of 1M lithium hydroxide solution (1.2 equiv.) was added dropwise, and the solution then stirred overnight at ambient temperature. The reaction was then examined by LC-MS, which typically showed clean formation of the free acid, with only a trace of ester remaining. The reaction was concentrated down under vacuum to remove THF, and more water added; the stirred solution was treated dropwise with 1M hydrochloric acid to give pH˜1, and the solution then equilibrated with ethyl acetate; the aqueous layer was removed, and the ethyl acetate layer washed with brine, dried over magnesium sulphate, filtered, and evaporated under vacuum. The residual oil was transferred to a weighed vial in a little ethyl acetate, and solvent removed under a stream of nitrogen; the sample was then placed in a drying pistol and pumped on overnight at 30° C./1 mbar.

¹H NMR (CDCl₃) confirmed the structure of the product as the free acid, and typically showed the presence of only a trace of methyl ester; a low level impurity, thought to be the cis-isomer, was always present, as was residual ethyl acetate.

The chiral purity of the product was assessed by re-esterifying a small sample of each of the four separated acid isomers and then analysing the esters by analytical chiral HPLC. About 5 mg of acid was dissolved in ether and treated with a freshly prepared solution of diazomethane in ether, to give a permanent yellow colour. After standing for 30 minutes at ambient temperature the solution was blown to dryness under nitrogen and re-dissolved in ethanol for chiral HPLC. The conditions used for the analysis were; analytical ChiralPak AD column (25 cm by 0.46 cm), 100% ethanol as stationary phase, flow rate of 0.25 ml/min UV detection (230 nm) at ambient temperature. Typical retention times for each isomer were: Peak 1, mixture 1 acid: 23.5 min; Peak 2, mixture 1 acid: 56 min; Peak 1, mixture 2 acid: 23.7 min; Peak 2, mixture 2 acid: 35 min. The chiral purity of each sample was essentially 100%.

### Example 4

### Assignment of the absolute stereochemistry of four stereoisomers of trans-2-[4-(1-hydroxyhexyl)phenyl]-5-oxo-cyclopentaneheptanoic acid

The absolute stereochemistry of AH13205 stereoisomers was determined by assigning the absolute configuration of the acid side chain-cyclopentanone junction using circular dichroism. The four stereoisomers of AH-13205, which all have the trans-configuration of the two side chains on the cyclopentanone have been separated (peak 1, mixture 1 acid; peak 2, mixture 1 acid; peak 1, mixture 2 acid and peak 2, mixture 2 acid); these are oils at room temperature.

To aid in assigning the absolute configuration, standards were also analysed. These are prostaglandin E₂ (PGE₂), (S)-1-(4-bromophenyl)hexan-1-ol (S-BPH) and (R)-1-(4-bromophenyl)hexan-1-ol (R-BPH) (shown below).

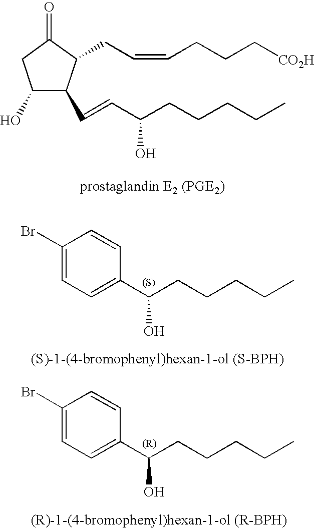

The samples were stored at room temperature and were dissolved in 100% ethanol and diluted to the concentrations as shown in table 1 prior to analysis.

TABLE 1

| | | |
|---|---|---|
| | | Final |
| Sample | Mass | concentration |

| | | | | |
|---|---|---|---|---|
| Peak 1, mixture 1 acid | 15.7 | mg | 19 | mg/mL |
| Peak 2, mixture 1 acid | 10.6 | mg | 19 | mg/mL |
| Peak 1, mixture 2 acid | 7.1 | mg | 19 | mg/mL |
| Peak 2, mixture 2 acid | 28.5 | mg | 19 | mg/mL |
| PGE₂ | Approximately 5 | mg | 0.7 | mg/mL |
| R-BPH | 15 | mg | 0.7 | mg/mL |
| S-BPH | 20 | mg | 0.7 | mg/mL |

A 1.0 cm pathlength quartz cuvette was used for the analysis of PGE₂, R-BPH and S-BPH. A 0.1 cm pathlength quartz cuvette was used for the analysis of the four AH13205 stereoisomers. Ethanol volumes were measured with a Gilson micropipette, for which they are in calibration if used quickly.

Instrument Calibration and Acceptance Criteria

The wavelength accuracy calibration criteria were met. Analysis of 0.06% (w/v) aqueous ammonium d-10-camphor sulfonate (ACS) showed signal intensity within specification, therefore no scaling factors were necessary.

The corresponding water baseline was subtracted from each sample spectrum, and the spectra were zeroed in the 360-400 nm region, which was outside the absorption band.

Results

Single analyses of UV and CD were undertaken for all samples. The UV and CD baseline subtracted and zeroed data for the standards PGE₂ and R-/S-BPH are shown in FIGS. 1a, 1b, 2a and 2b. The data for the four stereoisomers of AH13205 is shown in FIGS. 3a and 3b. The results are summarised in Table 2.

TABLE 2

| | | | | | | | UV | | CD |
|---|---|---|---|---|---|---|---|---|---|
| | Final | | | | First UV | | intensity | First CD | intensity |
| Sample | concentration | | Pathlength | | band/nm | | maximum | band/nm | maximum |
| Peak 1 | 19 | mg/mL | 0.1 | cm | 263.5/287.0 | | 1.42/0.28 | 297.0 | −630.5 |
| Mixture 1 | | | | | | | | | |
| acid | | | | | | | | | |
| Peak 2 | 19 | mg/mL | 0.1 | cm | 263.5/287.0 | (sh) | 1.17/0.23 | 297.0 | 511.5 |
| Mixture 1 | | | | | | | | | |
| acid | | | | | | | | | |
| Peak 1 | 19 | mg/mL | 0.1 | cm | 263.5/287.0 | | 1.59/0.33 | 297.0 | −685.0 |
| Mixture 2 | | | | | | | | | |
| acid | | | | | | | | | |
| Peak 2 | 19 | mg/mL | 0.1 | cm | 263.5/287.0 | | 1.34/0.28 | 297.0 | 583.5 |
| Mixture 2 | | | | | | | | | |
| acid | | | | | | | | | |
| PGE₂ | 0.7 | mg/mL | 1 | cm | 284.5 | | 0.13 | 299 | −239 |
| R-BPH | 0.7 | mg/mL | 1 | cm | 267.0 | | 0.74 | 269 | −5.4 |
| S-BPH | 0.7 | mg/mL | 1 | cm | 267.0 | | 0.76 | 269 | 6.46 |

The stereoisomeric samples (peak 1, mixture 1 acid; peak 2, mixture 1 acid; peak 1, mixture 2 acid and peak 2, mixture 2 acid) have two or three transitions of interest, one due to the n→π* transition at ˜290 nm and one due to the aromatic ring π→π* transition at about 260 nm and the next transition below this region. PGE₂ was used as a model for the n→π* part of the molecule and R-/S-BPH to model the π→π* part.

The π→π* absorbances were found to be weak and did not interfere with the analysis of the n→π* band.

The induced CD for PGE₂ is expected to be dominated by the acid group chain α- to the carbonyl. Analysis using the standard octant rule shows that this chain lies in the −z, +y and −x octant thus making −xyz (and therefore the expected CD curve) negative. This was observed experimentally (Table 2). Peak 1, mixture 1 acid and peak 1, mixture 2 acid samples showed negative CD curves and therefore have the same absolute stereochemistry at the side chain junction as PGE₂. Peak 2, mixture 1 acid and peak 2, mixture 2 acid samples showed positive CD curves and therefore have the opposite absolute stereochemistry from PGE₂ at this junction.

Both by pattern-matching with PGE₂ and with the octant rule correlation, the peak 1, mixture 1 acid and peak 1, mixture 2 acid samples have been shown to have the same absolute configuration as PGE₂ at the carbon α- to the carbonyl which is the point of attachment of the acid side chain to the cyclopentanone. The peak 2, mixture 1 acid and peak 2, mixture 2 acid samples have the opposite configuration to PGE₂ at this point. These results are shown in table 3.

TABLE 3

| | |
|---|---|
| | (1) |
| 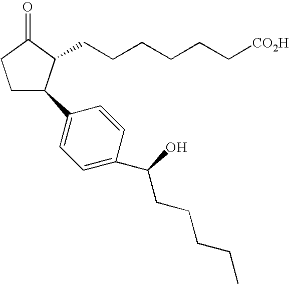 | |
| | (2) |
| 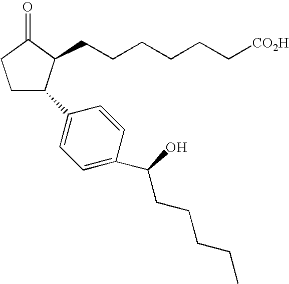 | |
| | (3) |
| 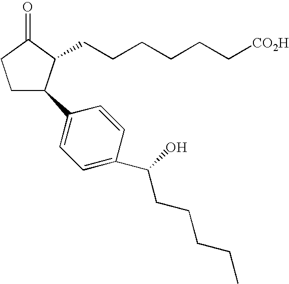 | |
| | (4) |
| 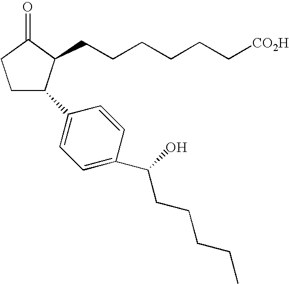 | |

| | Stereoisomer |
|---|---|
| Peak 1, Mixture 1 acid | (1) or (3) |
| Peak 2, Mixture 1 acid | (2) or (4) |
| Peak 1, Mixture 2 acid | (1) or (3) |
| Peak 2, Mixture 2 acid | (2) or (4) |

The determination of absolute stereochemistry by these CD studies is in agreement with the expected results of the stereoselective synthesis described for compound 17 [RSS], as predicted by comparison with the results from the literature of related stereoselective syntheses using compound (22).

As the stereochemistry at the 1-position of the hexyl chain in the 4-(1-hydroxyhexyl)phenyl side chain is known from the starting materials, the assignements in table 3 allow the stereochemistry of each of the isomers to be determined. These results are shown below.

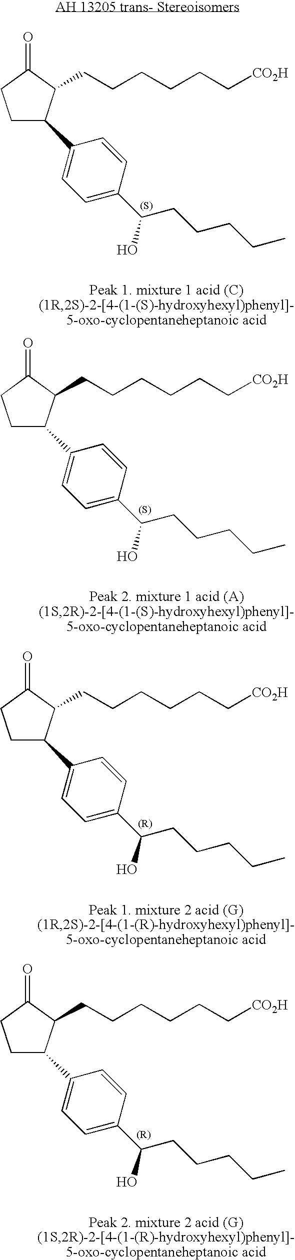

### Example 5

### Stereoselective preparation of (1R,2S)-2-[4-(1-(S)-hydroxyhexyl)phenyl]-5-oxo-cyclopentaneheptanoic acid (peak 1, mixture 1 acid)

### (a) Preparation of (1R, 2S)-2-{4-[1-(S)-tert-butyldimethylsilyloxy)hexyl]phenyl}-5-oxo-cyclopentanecarboxylic acid, (1R,2S,3R,4S)-{3-N-benzenesulfonyl-N-(3,5-dimethylphenyl)amino]-2-bornyl}ester (25)

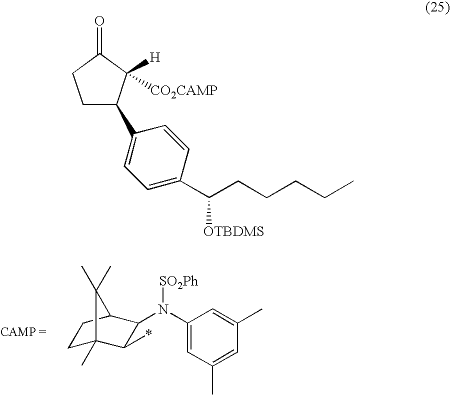

To a solution of (S)-1-(4-bromophenyl)-1-(tert-butyldimethyl-silyloxy)hexane (1.8 g) in anhydrous THF (24 ml) was added at −78° C. tert-butyllithium (1.7M in pentane; 5.6 ml) keeping the temperature below −65° C. After stirring for 2 hours at 78° C., lithium-2-thienylcyanocuprate (0.25M in THF; 19.2 ml) was added and the mixture left at −78° C. for an hour. A solution of 5-oxo-cyclopentenecarboxylic acid, (1R,2S,3R,4S)-{3-[N-benzenesulfonyl-N-(3,5-dimethylphenyl)amino]-2-bornyl}ester (1.7 g) in anhydrous THF (15 ml) was then added and the resulting mixture left at −78° C. for 75 minutes.

Saturated ammonium chloride solution (15 ml) was then added and the mixture allowed to warm up to 10-15° C. over the next hour. Further saturated ammonium chloride solution was added and the mixture extracted twice with ethyl acetate. The combined organic extracts were washed with ammonium chloride solution and subsequently with brine then dried over sodium sulphate and evaporated in vacuo. (1R,2S)-2-{4-[1-(S)-(tert-butyldimethylsilyloxy)hexyl]phenyl}-5-oxo-cyclopentanecarboxylic acid, (1R,2S,3R,4S)-{3-[N-benzenesulfonyl-N-(3,5-dimethylphenyl)amino]-2-bornyl}ester (1.8 g) was obtained as a foam following silica-gel column chromatography of the residue in 3:1 petroleum ether:diethyl ether.

¹H NMR (CDCl₃, δ): (a mixture of keto and enol forms) −0.3, −0.15, −0.1, 0.0, 0.1, 0.35, 0.45, 0.55 (8 s); 0.75-2.8 (c); 3.7 (m), 4.1 (m); 4.55 (m); 5.25 (dd); 5.5-5.8 (2 br s); 6.8(d); 6.95-7.5 (c); 11.0 (enol proton, s).

Mass Spectrum (m/z) ES⁺: 836.6 (M+Na)⁺

### (b) Preparation of (1R, 2S)-2-{4-[1-(S)-tert-butyldimethylsilyloxy)hexyl]phenyl}-5-oxo-cyclopentanecarboxylic acid, methyl ester (26)

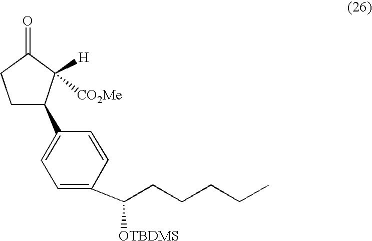

A solution of (1R,2S)-2-{4-[1-(S)-(tert-butyldimethylsilyloxy)hexyl]phenyl}-5-oxo-cyclopentanecarboxylic acid, (1R,2S,3R,4S)-{3-[N-benzenesulfonyl-N-(3,5-dimethylphenyl)amino]-2-bornyl} ester (1.1 g) in anhydrous methanol (60 ml) was heated to 150° C. in a sealed tube for 3 hours. After evaporation of the solvent in vacuo, (1R,2S)-2-{4-[1-(S)-(tert-butyldimethylsilyloxy)hexyl]phenyl}-5-oxo-cyclopentanecarboxylic acid, methyl ester (390 mg) was obtained as an oil following silica-gel column chromatography of the residue in 2:1 dichloromethane:petroleum ether.

¹H NMR (CDCl₃, δ): keto form −0.2 (3H, s); 0.0 (3H, s); 0.85 (9H, s); 0.85 (3H, t); 1.1-1.7 (8H, c); 1.95 (1H, c); 2.5 (3H, c); 3.35 (1H, d); 3.7 (3H, s); 3.8 (1H, m); 4.6 (1H, t); 7.15 (2H, d); 7.25 (2H, d).

Mass Spectrum (m/z) ES⁺: 611.6 (M+Na)⁺

### (c) Preparation of (2S)-1-methoxycarbonyl-2-{4-[1-(S)-(tert-butyldimethylsilyloxy)hexyl]phenyl}-5-oxo-cyclopentaneheptanoic acid, ethyl ester (27)

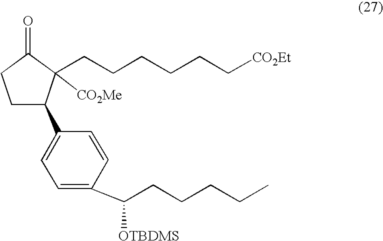

To a solution of (1R,2S)-2-{4-[1-(S)-(tert-butyldimethylsilyloxy)hexyl]phenyl}-5-oxo-cyclopentanecarboxylic acid, methyl ester (390 mg) in anhydrous DME (3.5 ml) was added sodium hydride (60% dispersion in oil; 39 mg). After leaving for an hour, ethyl 7-bromoheptanoate (0.3 ml) and a catalytic amount of sodium iodide were added and the mixture heated to reflux for 20 hours.

After cooling, the mixture was added to ammonium chloride solution and extracted twice with dichloromethane. The combined organic extracts were dried over sodium sulphate and evaporated in vacuo. The residue was taken up in anhydrous chloroform (10 ml) and a few crystals of para-toluenesulphonic acid monohydrate were added.

After stirring for an hour, sodium bicarbonate solution was added and the organic layer separated, dried over sodium sulphate and evaporated in vacuo. (2S)-1-methoxycarbonyl-2-{4-[1-(S)-(tert-butyldimethylsilyloxy)hexyl]phenyl}-5-oxo-cyclopentaneheptanoic acid, ethyl ester (290 mg) was obtained as an oil following silica-gel column chromatography of the residue in 0-5% diethyl ether in dichloromethane.

¹H NMR (CDCl₃, δ): −0.2 (3H, s); 0.0 (3H, s); 0.85 (9H, s); 0.85 (3H, t); 1.1-1.8 (20H, c); 2.0 (1H, c); 2.3 (4H, c); 2.6 (2H, c); 3.4 (3H, s); 3.45 (1H, m); 4.15 (2H, q); 4.6 (1H, t); 7.15 (2H, d); 7.25 (2H, d).

Mass Spectrum (m/z) ES⁺: 455.4 (M+Na)⁺

### (d) Preparation of (1R,2S)-2-{4-[1-(S)-(tert-butyldimethylsilyloxy)hexyl]phenyl}-5-oxo-cyclopentaneheptanoic acid (28)

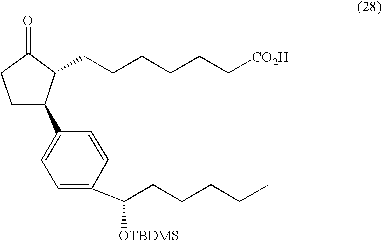

A mixture of (2S)-1-methoxycarbonyl-2-{4-[1-(S)-(tert-butyldimethylsilyloxy)hexyl]phenyl}-5-oxo-cyclopentaneheptanoic acid, ethyl ester (290 mg) and lithium iodide hydrate (650 mg) in 2,4,6-collidine (4 ml) was heated to reflux for 3 hours.

After cooling the mixture was added to ethyl acetate and washed twice with dilute hydrochloric acid, brine, dried over sodium sulphate and evaporated in vacuo. (1R,2S)-2-{4-[1-(S)-(tert-butyldimethylsilyloxy)hexyl]phenyl}-5-oxo-cyclopentaneheptanoic acid (190 mg) was obtained as an oil following silica-gel column chromatography of the residue in 4:1 petroleum ether:ethyl acetate.

¹H NMR (CDCl₃, δ): −0.2 (3H, s); 0.0 (3H, s); 0.85 (9H, s); 0.85 (3H, t); 1.0-2.3 (24H, c); 2.45 (1H, c); 2.9 (1H, m); 4.6 (1H, t); 7.15 (2H, d); 7.25 (2H, d).

Mass Spectrum (m/z) ES⁺: 525.4 (M+Na)⁺

### (e) Preparation of (1R,2S)-2-[4-(1-(S)-hydroxyhexyl)phenyl]-5-oxo-cyclopentaneheptanoic acid (peak 1, mixture 1 acid)

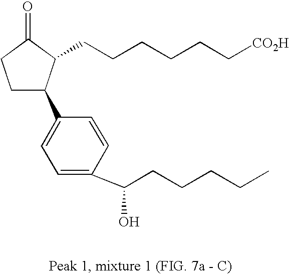

A solution of (1R,2S)-2-{4-[1-(S)-(tert-butyldimethylsilyloxy)hexyl]phenyl}-5-oxo-cyclopentaneheptanoic acid (190 mg) in THF (4 ml) and 2M hydrochloric acid (1.1 ml) was stirred for 20 hours at 30° C. The mixture was added to water and extracted twice with dichloromethane. The combined organic layers were dried over sodium sulphate and evaporated in vacuo. (1R,2S)-2-[4-(1-(S)-hydroxyhexyl)phenyl]-5-oxo-cyclopentaneheptanoic acid (115 mg) was obtained as an oil following silica-gel column chromatography of the residue in 5:2 petroleum ether:ethyl acetate.

¹H NMR (CDCl₃, δ): 0.85 (3H, t); 1.0-2.3 (24H, c); 2.45 (1H, c); 2.9 (1H, m); 4.6 (1H, t); 7.25 (2H, d); 7.35 (2H, d).

Mass Spectrum (m/z) ES⁻: 387.3 (M−H)⁻

[α]²⁵ −416.0 (365 nm), −139.5 (436 nm), −54.9 (546 nm), −45.6 (578 nm), −42.4 (589 nm) (c=0.51, CHCl₃; path length 100 mm)

### Example 6

### EP Binding and Agonism

The ability of compounds to bind to the human EP₂ receptor and their selectivity against all other EP receptors can be demonstrated in radioligand competition displacement binding experiments using cell lines stably transfected with the human EP receptors. The ability of compounds to stimulate the EP₂ receptor can be demonstrated in a second messenger cAMP functional assay, in primary human lymphocytes, monocytes or in human myometrium.

Test Details

Binding Ability to Human EP Receptors

Membranes were prepared from cells stably transfected with human EP receptor cDNA. In brief, cells were cultured to confluency, scraped from culture flasks, and centrifuged (800 g, 8 minutes, 4° C.). Cells were twice washed in ice cold homogenisation buffer containing 10 mMTris-HCl, 1 mM EDTA.2Na, 250 mM sucrose, 1 mM PMSF, 0.3 mM indomethacin, pH 7.4, homogenised and re-centrifuged as before. The supernatant was stored on ice and pellets re-homogenised and re-spun. Supernatants were pooled and centrifuged at 40000 g, 10 minutes, 4° C. Resultant membrane pellets were stored at −80° C. until use.

For assay, membranes expressing human EP₄, EP₃, EP₂ or EP₁ receptors were incubated in Millipore (MHVBN45) plates containing assay buffer, radiolabelled [³H]PGE₂ and 0.1 to 10 000 nM concentrations of compounds. Incubations were performed at suitable temperatures and for suitable times to allow equilibrium to be reached. Non-specific binding was determined in the presence of 10 uM PGE₂. Bound and free radiolabel was separated by vacuum manifold filtration using appropriate wash buffers, and bound radiolabel was determined by scintillation counting. Constituents of each of the buffers are included in table 4 below.

The affinity or pKᵢ of each compound for each receptor was calculated from the concentration causing 50% radioligand displacement (IC₅₀) using the Cheng-Prusoff equation:

$$Ki = \frac{{IC}_{50}}{1 + ( \frac{radioligand concentration}{radioligand KD} )}$$

This approach follows that set out in Kenakin, T. P., Pharmacologic analysis of drug receptor interaction. Raven Press, New York, 2^{nd} edition.

TABLE 4

| | Receptor | | | | | | | |
|---|---|---|---|---|---|---|---|---|
| | EP₁ | | EP₂ | | EP₃ | | EP₄ | |
| Protein/well | 6.5 | μg | 8 | μg | 5 | μg | 5 | μg |
| Final | 3.6 | nM | 3 | nM | 2.5 | nM | 1 | nM |
| [³H-PGE₂] | | | | | | | | |

| | | | | | |
|---|---|---|---|---|---|
| Buffer | Assay | 10 mM MES pH 6.0; | 10 mM MES | 10 mM MES pH | 10 mM MES |
| | | 10 mM MgCl₂; 1 mM | pH 6.0; 10 mM | 6.0; 10 mM | pH 6.0; 10 mM |
| | | EDTA, 3 uM | MgCl₂; 1 mM | MgCl2; 1 mM | MgCl₂; 1 mM |
| | | Indomethacin | EDTA | EDTA, 100 uM | EDTA, 3 uM |
| | | | | GTP-gamma-S | Indomethacin |
| | Wash | 10 mM MES pH 6.0; | 10 mM MES | 10 mM MES pH | 10 mM MES |
| | | 10 mM MgCl₂ | pH 6.0; 10 mM | 6.0; 10 mM MgCl₂ | pH 6.0; 1 mM |
| | | | MgCl₂ | | EDTA |

Effect of Compounds on Cyclase Production

The following describes an in vitro assay to determine the effect of compounds on cyclase production, that is, to determine their functional efficacy at the EP₂ receptor.

Cell Stimulation

HEK cells stably expressing the human EP₂ receptor were used for these assays. HEK-EP₂ cells were cultured in 96-well, poly-L-lysine coated plates at a density of 50,000 cells/well, and grown to confluence in humidified 95% O₂/5% CO₂ at 37° C. Culture medium was DMEM supplemented with 10% foetal bovine serum, 100 U/ml penicillin, 100 ng/ml streptomycin, 2.5 □g/ml fungizone, 2 mM glutamine, 250 □g/ml geneticin and 200 □g/ml zeocin.

On reaching confluence, culture media was rinsed off using DMEM with no additions, before 175 □l assay buffer (DMEM containing 1 mM 3-isobutyl-1-methylxanthine and 3 □M indomethacin) was added to each well. This was allowed to incubate for 1 hr before the cells were stimulated with the test compounds (in triplicate) at final concentrations of 10⁻⁹M to 10⁻⁵M for 15 minutes. The assay was terminated by the addition of 25 □l 1M hydrochloric acid. Plates were then frozen for a minimum of 12 hours or until required for radioligand displacement assay.

Radioligand Displacement Assay

Plates were thawed quickly at 37° C., and neutralised with 25 □l 1M sodium hydroxide. 30 □l of supernatant was transferred to 96-well Millipore (MAFNOB) plates coated with 0.1% Polyethylenimine. These supernatants were diluted by addition of 90□1 cAMP assay buffer (50 mM Tris, 5 mM EDTA, pH 7.0). A cAMP standard curve (10⁻¹¹M to 10⁻⁵M) was constructed. 15 □l of 3′:5′-cAMP-dependent protein kinase (final concentration 8 □g/well), and 15 □l [³H]-cAMP (final concentration 2 nM/well) were added to each well.

Plates were incubated on ice for 2 hours, before bound and free radiolabel were separated by vacuum filtration harvesting on the Millipore manifold, using ice cold water as the termination buffer. Filter plates were allowed to dry overnight, before addition of 50 □l Microscint. Radioactivity was determined using the Microbeta Trilux scintillation counter. cAMP accumulation was determined from the standard curve, and the values plotted as pmoles cAMP/well.

Effect of Compounds on Human Myometrial Activity

The following describes an in vitro functional assay, using human myometrial smooth muscle, to determine the affinity of the test compounds at the EP₂ receptor in human tissues.

Sections of human myometrium were prepared from samples of surgically removed uterus longitudinal myometrial muscle strips (2 mm wide by 10 mm long) were then cut and suspended between stainless steel hooks in organ chambers containing oxygenated (95% O₂/5% CO₂) Krebs solution at 37° C. The composition of the Krebs solution was as follows: NaCl (118.2 mM), KCl (4.69 mM), MgSO₄.7H₂O (1.18 mM), KH₂PO₄ (1.19 mM), glucose (11.1 mM), NaHCO₃ (25.0 mM), CaCl₂.6H₂O (2.5 mM), indomethacin 3×10⁻⁶M.

Tissues were placed under a tension equivalent to 25 mN and left overnight at room temperature. The following day the tissues were maintained at 37° C., washed and placed under a tension of 15 mN then allowed to equilibrate for a period of at least 30 minutes. Responses were recorded using isometric transducers coupled to an Apple Macintosh computer via a MacLab interface. After 60 minutes, the muscle sections of the human myometrium were stimulated electrically (15 ms pulse width, for 10 s every 100 s at 15V and 0.5-40 Hz) using parallel platinum wire electrodes and a Multistim D330 pulse stimulator. Upon electrical stimulation, the strips of human myometrial smooth muscle responded with a rapid contraction. Once the response to electrical stimulation had stabilised (stimulated responses differed by no more than 10%), the strips were exposed to increasing concentrations of test compounds (1×10⁻⁷ to 1×10⁻⁴M, incubated for at least 15 minutes at each concentration). At the end of the experiment, application of sodium nitroprusside (SNP, a nitric oxide donor that causes smooth muscle relaxation) (1×10⁻⁴M) was used to produce a standard relaxatory response. To determine the affinity of the compounds, the concentration of test compound required to produce half-maximal effects (EC₅₀) was calculated, as was the maximum response (calculated as a percentage of the standard response produced with SNP).

Results

Binding Ability to Human EP Receptors

In these tests, the affinity of the four separated stereoisomers of AH-13205 were determined, and the results are shown in FIG. 4 (data is shown as mean±s.e for 4 experiments). The stereoisomer isolated in peak 1 of mixture 1 was shown to be the most potent, having a pKi of 7.1.

The full results of the binding tests are shown in table 5 as pKi values:

TABLE 5

| Compound | EP₂ | EP₁ | EP₃ | EP₄ |
|---|---|---|---|---|
| Peak 1, Mixture 1 acid | 7.1 | — | 5.7 | 5.0 |
| Peak 2, Mixture 1 acid | 5.8 | — | 4.8 | 4.5 |
| Peak 1, Mixture 2 acid | 6.9 | — | 4.8 | 5.1 |
| Peak 2, Mixture 2 acid | 6.3 | — | 4.7 | 4.8 |
| AH-13205 | 6.4 | 5.0 | 5.2 | 4.6 |

From this table, it can be seen that Peak 1, mixture 2 acid is the most selective of the stereoisomers.

Effect of Compounds on CAMP Production

In these tests, the effect of the separated stereoisomers and AH-13205 on cAMP production mediated by human EP receptor stimulation was assessed. All the compounds showed the same maximal response, but their potency differed, as shown in FIG. 5 and table 6 (data is shown as mean±s.e. for 4 experiments).

TABLE 6

| Compound | Mean Log (EC₅₀) | S.E.M. | Mean EC50 (nM) |
|---|---|---|---|
| Peak 1, Mixture 1 acid | −8.01 | 0.22 | 10 |
| Peak 2, Mixture 1 acid | −6.49 | 0.19 | 323 |
| Peak 1, Mixture 2 acid | −7.25 | 0.19 | 56 |
| Peak 2, Mixture 2 acid | −6.39 | 0.24 | 407 |
| AH13205 | −7.49 | 0.29 | 32 |

Effect of Compounds on Human Myometrial Activity

Application of AH-13205 was shown to inhibit electrically-induced contractions in human myometrium—points A, B and C correspond to the addition of increasing amounts of AH-13205 (10⁻⁶, 10⁻⁵ and 10⁻⁴ M) (FIG. 6). The potency of the effect was in accordance with interaction at a prostaglandin EP₂ receptor, as the vehicle containing AH-13205 was shown to have no effect (FIG. 7).

The effects of the two most potent stereoisomers (Peak 1, Mixture 1 acid and Peak 1, Mixture 2 acid) were investigated, and compared to the effects of AH-13205. Peak 1, Mixture 1 acid, Peak 1, Mixture 2 acid and AH13205, all caused concentration-dependent inhibition of the EFS-evoked response. The pEC50 s were 5.9±0.2 (n=7), 5.3±0.1 (n=6) and 5.3±0.2 (n=7) (FIG. 8). There was no significant differences between the maximum inhibitory effects observed, with inhibition of EFS-induced contractions of 56±5% (Peak 1, Mixture 1 acid), 57±2% (Peak 1, Mixture 2 acid) and 49±5% (AH-13205). SNP caused further inhibition on top of the compounds, equivalent to 60-70% of the control EFS response. The SNP inhibitory effect was reversed over a 60-80 minutes washing period but the inhibitory effects of the compounds tested were not.

In addition, the effect of terbutaline, a β adrenoceptor agonist, on EFS-induced contractions of myometrium was investigated, and shown to have no significant inhibitory effect on the EFS-evoked contractions (98±5% of the control EFS-induced contraction at 10⁻⁴M, n=7 donors).

### Example 7

### Inhibition of IL-2, TNFα and IFN-γ Production

Lymphocytes are mononuclear leukocytes, which participate in specific immune responses to foreign antigens and in the manifestation of auto-immune diseases. T lymphocytes produce IL-2, a key factor for lymphocyte activation and proliferation, in response to antigen stimulation via the CD3-T cell receptor complex and the pathway involved in this response is the NF-AT. This response can be demonstrated in vitro by using selective monoclonal antibodies with specificity to the CD3 molecules on T cells. A lymphocyte assay was designed to model this response and to determine the effect of test compound on IL-2 production by anti-CD3-stimulated T cells isolated from peripheral blood. This assay uses a sub-optimal dose of an anti-CD3 monoclonal antibody (OKT3, 25 ng/ml) immobilised to a 96-well plate to stimulate a T cell response. The level of IL-2 released into the cell culture supernatants was quantified using a standard sandwich ELISA. Similarly, other cytokines such as TNFα and IFN-γ can also be measured in the same assay. The assay can be extended to 72-hour time point when lymphocyte proliferation in response to anti-CD3 antibody can be observed, hence the effect of immune modulatory compounds examined.

Monocytes are peripheral mononuclear phagocytes that participate in inflammatory responses. TNFα production by monocytes plays an important role in inflammatory responses and can cause considerable tissue damage if the level remained unchecked. Inhibition of TNFα secretion by activated monocytes may provide an attractive therapy for the treatment of inflammatory conditions.

One of the most potent microbial triggers of TNFα release by monocytes is lipopolysaccharide (LPS) and this response is via the NF-KB pathway. A 96 well in vitro assay was established to determine the effects of test compounds on LPS-induced TNFα secretion by human peripheral blood monocytes. The level of TNFα in assay supernatants was quantified using a standard sandwich ELISA.

Test Details

Human peripheral blood mononuclear cells from healthy volunteers were isolated from whole blood by Ficoll-Hypaque density centrifugation and adherence to plastic. The non-adherent lymphocyte fraction was used to set up the lymphocyte assay and the adherent monocytes were then recovered by scraping and subsequently used in the monocyte assay.

Lymphocyte Assay

Lymphocytes were then seeded to a 96-well plate pre-coated with anti-CD3 monoclonal antibody (OKT3) at 25 ng/ml and immediately, the test compounds (Peak 1, mixture 1 acid; Peak 1, mixture 2 acid; AH-13205 racemate; PGE₂) in appropriate dilutions were added to corresponding wells according to the experimental design. The plate was incubated for 24 hours at 37° C. with 5% CO₂ in air and supernatants were recovered for ELISA analysis at the end of incubation period. The levels of IFN-γ was assessed by using the ProteoPlex 16 well human cytokine array assay kit according to the manufacturer's instruction.

For the measurement of lymphocyte proliferation driven by immobilised anti-CD3 monoclonal antibody, the assay was set up in the same way as for the measurement of IL-2 release, except that the cells were cultured for 72 hours in the presence or absence of test compounds. Four hours prior to the termination of the proliferation assay, a novel tetrazolium compound solution supplied by Promega in the format of Cell Proliferation Assay Kit was added to individual wells according to the manufacturer's instruction. The plate was then placed back in the incubator for the remaining 4 hours and the calorimetric reaction was measured using a spectrophotometer at an absorbent wavelength of 490 nm (SpectraMax, Molecular Devices) according to the manufacturer's instruction.

Monocyte Assay

For the monocyte assay, the cells were plated onto 96-well plates and pre-treated for 1 hour at 37° C./5% CO₂ with the test compound (Peak 1, mixture 1 acid; Peak 1, mixture 2 acid; AH-13205 racemate), followed by the addition of LPS (100 ng/ml) to initiate the reaction. The plate was incubated for 24 hours and supernatants were recovered for the measurement of TNFα production by ELISA.

Macrophage Assay

Human lung parenchyma was cut into small pieces and perfused with ice-cold phosphate buffered saline (PBS) to remove contaminating blood and mucus. The tissues were then chopped with scissors in the presence of Minimum Essential Medium supplemented with penicillin, streptomycin, L-glutamine and DNase (0.25 mg/ml). The chopped tissues were shaken gently to dislodge the macrophages. A crude cell suspension was then obtained by the removal of the tissues with a sterile filter (150 μm pore size). The resulting cell suspension was spun and the cell pellet collected. Contaminating red blood cells were depleted with a red blood cell lysis buffer and the remaining cells washed twice with PBS by centrifugation. Alveolar macrophages were then purified from this cell preparation by using a positive selection method for CD14-molecule bearing cells using a VarioMac™ Separator and respective positive selection reagents and columns supplied by Miltenyi Biotec Ltd according to the manufacturer's instruction.

For the assay, alveolar macrophages were resuspended in complete culture medium consisting of RPMI1640 supplemented with 10% foetal calf serum, L-glutamine and antibiotics. The cells were then plated into 24 well plates (4×10⁵ cell/well) and incubated overnight at 37° C. with 5% CO₂ in air to allow cell adherence. The exhausted medium was then removed from the plates and the plates rinsed briefly with fresh medium before the addition of test compound solutions. The test compounds were incubated with the cells for 30 minutes before the addition of E. Coli LPS (1 μg/ml). The assay plates were incubated in a humidified incubator at 37° C. with 5% CO₂ in air for 24 hours. The release of TNF_{α} by the cells into the culture supernatants was quantified using an ELISA kit (DuoSet® human TNF_{α} ELSIA Development System) supplied by R+D Systems (Europe) according to the manufacturer's instruction. Indomethacin at 3 μM was included in all treatments to inhibit the possible release of endogenous prostaglandin E₂.

Results

Lymphocyte Assay

FIG. 9 shows the results of IL-2 production by three test compounds given as mean of four donors (except peak 1, mixture 2 acid which was tested in one donor only). These results are summarized in table 7.

TABLE 7

| Compound | Mean Log (EC₅₀) | Mean EC50 (μM) |
|---|---|---|
| Peak 1, Mixture 1 acid | −6.006 | 0.986 |
| AH13205, racemate | −5.549 | 2.823 |
| PGE₂ | −7.554 | 0.028 |

These results show that EP₂ agonists concentration-dependently inhibit IL-2 production by OKT3 activated T cells. The order of potencies in the assay is PGE₂>Peak 1, Mixture 1 acid>AH13205 (racemate) according to their respective EC₅₀ values.

FIG. 10 shows the results given as mean of three to five donors of IL-2 production by three EP2 receptor agonists. These results are summarized in table 8.

TABLE 8

| Compound | Mean Log (EC₅₀) | Mean EC50 (μM) |
|---|---|---|
| Peak 1, Mixture 1 acid | −5.880 | 1.319 |
| Butaprost | −6.906 | 0.1242 |
| PGE₂ | −7.677 | 0.02105 |

These results show that EP₂ agonists concentration-dependently inhibit IL-2 production by OKT3 activated T cells. The order of potencies in the assay is PGE₂>butaprost>Peak 1, Mixture 1 acid according to their respective EC₅₀ values.

FIG. 11 shows the results from two donors of interferon gamma release by Peak 1, Mixture 1 acid. These results show that EP₂ agonists concentration-dependently inhibit interferon gamma release.

FIG. 12 shows the results given as mean of three donors of TNFα production by three EP2 receptor agonists. These results are summarized in table 9.

TABLE 9

| Compound | Mean Log (EC₅₀) | Mean EC50 (μM) |
|---|---|---|
| Peak 1, Mixture 1 acid | −5.632 | 2.332 |
| Butaprost | −6.535 | 0.2917 |
| PGE₂ | −7.971 | 0.01069 |

These results show that EP₂ agonists concentration-dependently inhibit TNFα production by lymphocytes. The order of potencies in the assay is PGE₂>butaprost>Peak 1, Mixture 1 acid according to their respective EC₅₀ values.

FIG. 13 shows the results given as mean of three donors of lymphocyte proliferation by three EP₂ receptor agonists. These results are summarized in table 10.

TABLE 10

| Compound | Mean Log (EC₅₀) | Mean EC50 (μM) |
|---|---|---|
| Peak 1, Mixture 1 acid | −5.000 | 9.995 |
| Butaprost | −6.015 | 0.996 |
| PGE₂ | −7.589 | 0.02579 |

These results show that EP₂ agonists concentration-dependently inhibit lymphocyte proliferation. The order of potencies in the assay is PGE₂>butaprost>Peak 1, Mixture 1 acid according to their respective EC₅₀ values.

Monocyte Assay

FIG. 14 shows the results given as mean of three donors. These results are summarized in table 11.

TABLE 11

| Compound | Mean Log (EC₅₀) | Mean EC50 (μM) |
|---|---|---|
| Peak 1, Mixture 1 acid | −5.749 | 1.783 |
| Peak 1, Mixture 2 acid | −5.172 | 6.730 |
| AH13205, racemate | −4.704 | 19.780 |

These results show that the EP₂ agonists concentration-dependently inhibited TNFα production by LPS-stimulated monocytes. The order of potencies based on their respective EC₅₀ values is Peak 1, Mixture 1 acid>Peak 1, Mixture 2 acid>AH13205 (racemate).

Macrophage Assay

FIG. 15 shows the results given as mean of three donors. These results are summarized in table 12.

TABLE 12

| Compound | Mean Log (EC₅₀) | Mean EC50 (μM) |
|---|---|---|
| Peak 1, Mixture 1 acid | −5.154 | 7.023 |
| PGE₂ | −6.904 | 0.1247 |

These results show that the EP₂ agonists concentration-dependently inhibited TNFα production by macrophages. The order of potencies based on their respective EC₅₀ values is PGE₂>Peak 1, Mixture 1 acid.

[image]

[image]

[image]

1. A compound selected from one of the following: 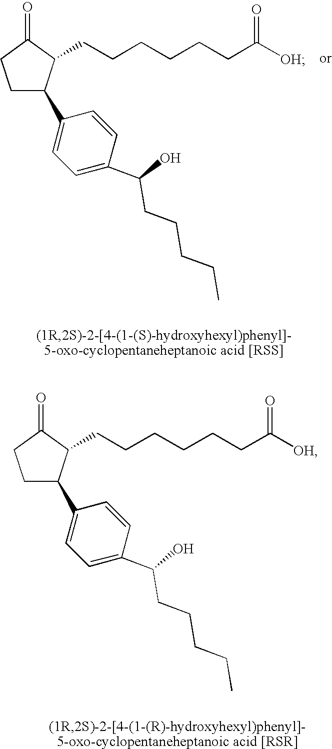
or a salt, solvate, chemically protected form or prodrug thereof.

2. (trans-2-[4-(1-hydroxyhexyl)phenyl]-5-oxo-cyclopentaneheptanoic acid, of which at least 90% by weight is selected from one of the following forms: 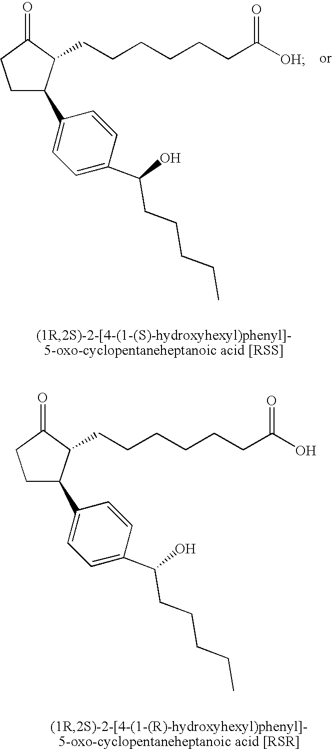
or a salt, solvate, chemically protected form or prodrug thereof.

3. 2-[4-(1-hydroxyhexyl)phenyl]-5-oxo-cyclopentaneheptanoic acid, of which at least 80% by weight is in one of the following forms: 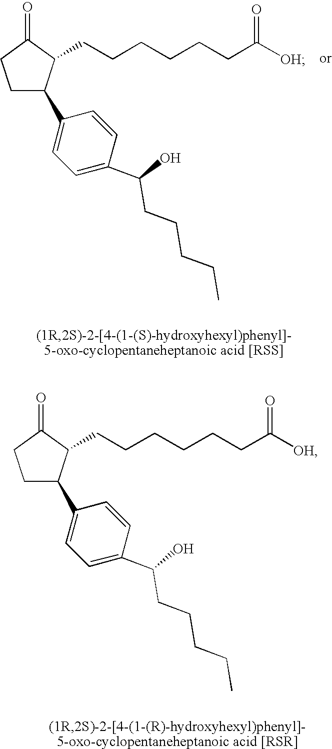
or a salt, solvate, chemically protected form or prodrug thereof.

4. A method of making a compound according to claim 1.

5. (canceled)

6. A pharmaceutical composition comprising a compound according to claim 1, or a pharmaceutically acceptable salt thereof, together with a pharmaceutically acceptable carrier or diluent.

7. A method of preparing a medicament comprising admixing a compound according to claim 1, or a pharmaceutically acceptable salt thereof with a carrier.

8-14. (canceled)

15. A method of treating a condition which can be alleviated by the inhibtion of:
(i) human T-cell activation (proliferation);
(ii) the release of IL-2;
(iii) the release of TNF_{α}; or
(iv) the release of IF_{γ}; or
a method of treatnig a condition which can be alleviated by agonism of an EP₂ receptor, or
a method of treating graucoma, or dysmenorrhoea or pre-term labour, or
a method of treating a psoriasis, or an inflammatory lung disease,
which method comprises administering to a patient in need of treatment an effective amount of an EP₂ receptor agonist, or a pharmaceutically acceptable salt thereof.

16-17. (canceled)

18. A method according to claim 15, wherein the EP₂ receptor agonist is a compound selected from one of the following: 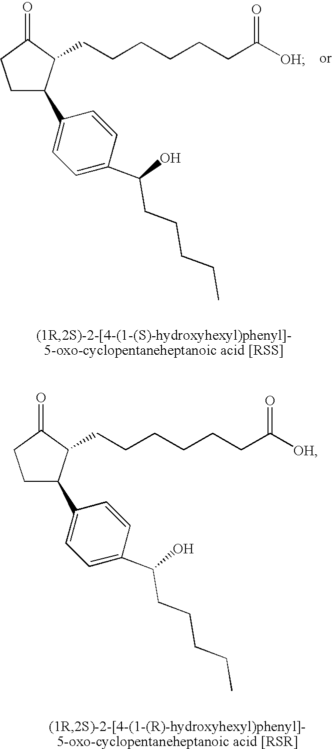
or a salt, solvate, chemically protected form or prodrug thereof.

19. A method according to claim 15, wherein the EP₂ receptor agonist is (trans-2-[4-(1-hydroxyhexyl)phenyl]-5-oxo-cyclopentaneheptanoic acid, of which at least 90% by weight is selected from one of the following forms: 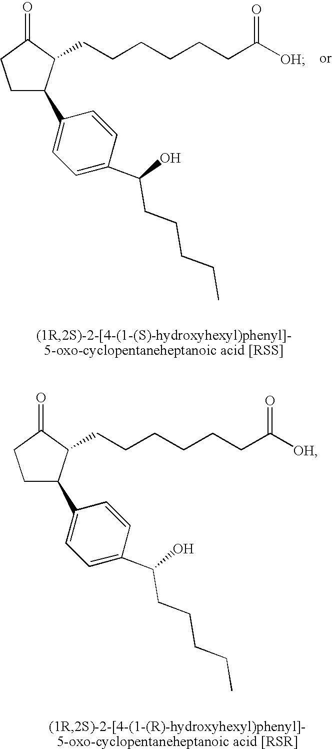
or a salt, solvate, chemically protected form or prodrug thereof.

20. A method according to claim 15, wherein the EP₂ receptor agonist is 2-[4-(1-hydroxyhexyl)phenyl]-5-oxo-cyclopentaneheptanoic acid, of which at least 80% by weight is in one of the following forms: 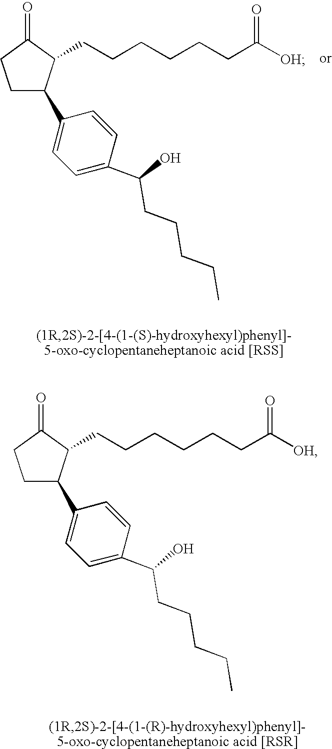
or a salt, solvate, chemically protected form or prodrug thereof.
